(19) United States
(12) Patent Application Publication
(10) Pub. No.: US 2021/0389329 A1
(43) Pub. Date: Dec. 16, 2021

(54) Compositions and Methods for Discriminating Infectious from Non-Infectious CNS Disorders

(71) Applicants: THOMAS JEFFERSON UNIVERSITY, Philadelphia, PA (US); THE CHILDREN'S HOSPITAL OF PHILADELPHIA, Philadelphia, PA (US)

(72) Inventors: Danielle FORTUNA, Blue Bell, PA (US); Mark Thomas CURTIS, Gladwyne, PA (US); Douglas Craig HOOPER, Medford, NJ (US); Timothy Chao, Philadelphia, PA (US); Ana María CÁRDENAS CAICEDO, Philadelphia, PA (US)

(21) Appl. No.: 17/289,499

(22) PCT Filed: Oct. 29, 2019
(86) PCT No.: PCT/US2019/058667

§ 371 Date: Apr. 28, 2021

Related U.S. Application Data
(60) Provisional application No. 62/751,810, filed on Oct. 29, 2018.

(51) Int. Cl. G01N 33/68; G01N 33/574; C12Q 1/6883; C12Q 1/6886; C12Q 1/6888

(52) U.S. Cl. CPC G01N 33/6869; G01N 33/57488; C12Q 1/6883; C12Q 1/6886; G01N 2800/28; G01N 2333/7155; G01N 2333/521; G01N 2800/26; C12Q 1/6888

(57) ABSTRACT

The present invention provides compositions and methods for discriminating infectious from non-infectious CNS disorders, and providing appropriate treatment thereof.

### CROSS-REFERENCE TO RELATED APPLICATIONS

The present application is entitled to priority under 35 U.S.C. § 119(e) to U.S. Provisional Patent Application No. 62/751,810, filed Oct. 29, 2018, which is incorporated herein by reference in its entirety.

### BACKGROUND OF THE INVENTION

Rapid identification of CNS disease is critical to implement prompt measures and initiate appropriate disease-specific treatments. CNS infections are major causes of morbidity and mortality, yet such infections may be amenable to therapeutic intervention if promptly diagnosed. Given that many pathogens worldwide are rapidly expanding their geographic ranges, certain infections may not be considered at the initial clinical presentation, creating a lag between clinical suspicion and diagnosis. Continuous advancement in therapeutic and preventive options, such as small molecule antiviral drugs, therapeutic antibodies, and new vaccines, makes early diagnosis of infection even more important. Initial identification of a CNS disorder as infectious and distinguishing it from other non-infectious pathologic processes, however, is not entirely straightforward. The differential diagnoses considered in a patient with symptoms of CNS disorder (e.g., acute mental status change, focal neurologic deficit, severe headache, and photophobia) are numerous and often include infection, autoimmune disorders, demyelinating disease and neoplasms. Importantly, therapy for these various diseases is drastically different. For example, immunosuppressive and immunomodulatory agents indicated in CNS autoimmune disease and demyelinating disorders (including multiple sclerosis) would be contraindicated and potentially detrimental in the setting of a CNS infection.

Due to its close anatomical relationship to critical structures in the CNS, the CSF serves as a convenient conduit for inflammatory mediators and signaling proteins released during changes in the CNS environment. The rapidly responding innate immune system is present and active in the CNS and is sensitive to a variety of alterations in CNS homeostasis. CSF, therefore, can be examined to understand the current state of immune activation. When activated in response to infection, tissue damage, or mass lesions, pro- and anti-inflammatory cytokines and growth factors are released and detectable in the CSF. It is known that pathogens, autoimmune processes, demyelination, and neoplasms target different CNS resident cells and activate innate immunity in different ways, resulting in distinct patterns of CSF cytokines which reflect the range of pathologies.

A need exists for novel compositions and methods for discriminating infectious from non-infectious CNS disorders. The present invention addresses and satisfies this need.

### SUMMARY OF THE INVENTION

In one aspect, the invention includes a method of treating a Central Nervous System (CNS) disorder in a pediatric patient in need thereof. The method comprises measuring cytokine levels in a sample from the pediatric patient, and comparing the patient sample levels to a first reference sample. When the level of IL17A is lower in the patient sample compared to the reference sample, the CNS disorder is not a bacterial infection, and wherein when the level of IL17A is higher in the patient sample compared to the reference sample, the CNS disorder is a bacterial infection and an anti-bacterial treatment is administered to the patient.

In certain embodiments, when the CNS disorder is not a bacterial infection, and the level of IL1RA is lower in the patient sample compared to the reference sample, and the level of IL1A is lower in the patient sample compared to the reference sample, the CNS disorder is an autoimmune disorder and a treatment for an autoimmune disorder is administered to the patient.

In certain embodiments, when the CNS disorder is not a bacterial infection, and the level of IL1RA is higher in the patient sample compared to the reference sample, and the level of IP10 is lower in the patient sample compared to the reference sample, and the level of IL1RA is lower in the patient sample compared to a second reference sample, the CNS disorder is an autoimmune disorder and a treatment for an autoimmune disorder is administered to the patient.

In certain embodiments, when the CNS disorder is not a bacterial infection, and the level of IL1RA is higher in the patient sample compared to the reference sample, and the level of IP10 is lower in the patient sample compared to the reference sample, and the level of IL1RA is higher in the patient sample compared to a second reference sample, the CNS disorder is a cancer and a treatment for cancer is administered to the patient.

In certain embodiments, when the CNS disorder is not a bacterial infection, and the level of IL1RA is higher in the patient sample compared to the reference sample, and the level of IP10 is higher in the patient sample compared to the reference sample, and the level of MDC is lower in the patient sample compared to the reference sample, the CNS disorder is viral and an anti-viral treatment is administered to the patient.

In certain embodiments, when the CNS disorder is not a bacterial infection, and the level of IL1RA is higher in the patient sample compared to the reference sample, and the level of IP10 is higher in the patient sample compared to the reference sample, and the level of MDC is higher in the patient sample compared to the reference sample, the CNS disorder is a cancer and a treatment for cancer is administered to the patient.

In another aspect, the invention includes a method of treating a Central Nervous System (CNS) disorder, comprising measuring cytokine levels in a sample from a patient and comparing the patient sample levels to a reference sample, wherein when the level of IP-10/CXCL10 is lower in the patient sample compared to the reference sample, the CNS disorder is non-infectious, and when the level of IP-10/CXCL10 is higher in the patient sample compared to the reference sample, the CNS disorder is infectious, and a treatment is administered to the patient that treats the infection.

In certain embodiments, when the CNS disorder is infectious and the level of MDC/CCL22 is higher in the patient sample compared to the reference sample, the CNS disorder is a non-viral disorder, and an anti-bacterial and/or anti-fungal and/or anti-parasitic treatment is administered to the patient, and when the level of MDC/CCL22 is lower in in the patient sample compared to the reference sample, the CNS disorder is a viral disorder, and an anti-viral treatment is administered to the patient.

In certain embodiments, when the CNS disorder is non-infectious, and the levels of IL-8 and GRO/CXCL1 are higher in the patient sample compared to the reference sample, the CNS disorder is a glioma, and a treatment for gliomas is administered to the patient, and when the level of IL-8 and GRO/CXCL1 are lower in the patient sample compared to the reference sample, the CNS disorder is an autoimmune disorder or a lymphoma, and a treatment for an autoimmune disorder or a lymphoma is administered to the patient.

In certain embodiments, when the CNS disorder is an autoimmune disorder or a lymphoma, and when the level PDGF-AA is higher in the patient sample compared to the reference sample, the CNS disorder is a lymphoma, and an treatment for lymphomas is administered to the patient, and when the level PDGF-AA is lower in the patient sample compared to the reference sample, the CNS disorder is an autoimmune disorder and a treatment for an autoimmune disorder is administered to the patient.

In another aspect, the invention includes a method of treating a Central Nervous System (CNS) disorder comprising measuring cytokine levels in a sample from a patient and comparing the patient sample levels to a reference sample, wherein when the level of IL-6 is lower in the patient sample compared to the reference sample, and the level of MDC is higher in the patient sample compared to the reference sample, the CNS disorder is an autoimmune disorder and a treatment for an autoimmune disorder is administered to the patient.

In another aspect, the invention includes a method of treating a Central Nervous System (CNS) disorder comprising measuring cytokine levels in a sample from a patient and comparing the patient sample levels to a first, second, and third reference sample. When the level of IL-6 is higher in the patient sample compared to the first reference sample, and the level of MDC is lower in the patient sample compared to the second reference sample and compared to the third reference sample, the CNS disorder is an autoimmune disorder and a treatment for an autoimmune disorder is administered to the patient.

In another aspect, the invention includes a method of treating a Central Nervous System (CNS) disorder comprising measuring cytokine levels in a sample from a patient and comparing the patient sample levels to a first, second, and third reference sample. When the level of IL-6 is higher in the patient sample compared to the first reference sample, and the level of MDC is lower in the patient sample compared to the second reference sample but higher compared to the third reference sample, the CNS disorder is a lymphoma and a treatment for lymphoma is administered to the patient.

In another aspect, the invention includes a method of treating a Central Nervous System (CNS) disorder comprising measuring cytokine levels in a sample from a patient and comparing the patient sample levels to a first, second, third, and fourth reference sample. When the level of IL-6 is higher in the patient sample compared to the first reference sample, and the level of MDC is higher in the patient sample compared to the second reference sample but lower compared to the third reference sample, and the level of MIP-1A is higher in the patient sample compared to the fourth reference sample, the CNS disorder is a cancer and a treatment for cancer is administered to the patient.

In another aspect, the invention includes a method of treating a Central Nervous System (CNS) disorder comprising measuring cytokine levels in a sample from a patient and comparing the patient sample levels to a first, second, third, and fourth reference sample. When the level of IL-6 is higher in the patient sample compared to the first reference sample, and the level of MDC is higher in the patient sample compared to the second reference sample but lower compared to the third reference sample, and the level of MIP-1A is lower in the patient sample compared to the fourth reference sample, the CNS disorder is a cancer and a treatment for cancer is administered to the patient.

In another aspect, the invention includes a method of treating a Central Nervous System (CNS) disorder comprising measuring cytokine levels in a sample from a patient and comparing the patient sample levels to a first, second, third, and fourth reference sample. When the level of IL-6 is higher in the patient sample compared to the first reference sample, and the level of MDC is higher in the patient sample compared to the second reference sample and the third reference sample, and the level of IL-8 is lower in the patient sample compared to the fourth reference sample the CNS disorder is a lymphoma and a treatment for lymphoma is administered to the patient.

In another aspect, the invention includes a method of treating a Central Nervous System (CNS) disorder comprising measuring cytokine levels in a sample from a patient and comparing the patient sample levels to a first, second, third, and fourth reference sample. When the level of IL-6 is higher in the patient sample compared to the first reference sample, and the level of MDC is higher in the patient sample compared to the second reference sample and the third reference sample, and the level of IL-8 is higher in the patient sample compared to the fourth reference sample, the CNS disorder is infectious and an anti-bacterial and/or anti-fungal and/or anti-parasitic treatment and/or anti-viral treatment is administered to the patient.

In another aspect, the invention includes a composition useful for determining whether a CNS disorder in a patient is infectious, wherein the composition comprises an agent capable of binding IP-10/CXCL10, and an agent capable of binding MDC/CCL22.

In another aspect, the invention includes a composition useful for determining whether a CNS disorder in a patient is a lymphoma, a glioma, or an autoimmune disorder, wherein the composition comprises an agent capable of binding IP-10/CXCL10, an agent capable of binding IL-8, an agent capable of binding GRO/CXCL1, and an agent capable of binding PDGF-AA.

In another aspect, the invention includes a composition useful for determining whether a CNS disorder in a pediatric patient is an autoimmune disorder, wherein the composition comprises an agent capable of binding IL17A, an agent capable of binding IL1RA, and an agent capable of binding IL1A.

In another aspect, the invention includes a composition useful for determining whether a CNS disorder in a pediatric patient is an autoimmune disorder, wherein the composition comprises an agent capable of binding IL17A, an agent capable of binding IL1RA, and an agent capable of binding IP10.

In another aspect, the invention includes a composition useful for determining whether a CNS disorder in a pediatric patient is bacterial, wherein the composition comprises an array comprising an agent capable of binding IL17A and an agent capable of binding IL1RA.

In another aspect, the invention includes a composition useful for determining whether a CNS disorder in a pediatric patient is viral, wherein the composition comprises an array comprising an agent capable of binding IL17A, an agent capable of binding IL1RA, an agent capable of binding IP10, and an agent capable of binding MDC.

In another aspect, the invention includes a composition useful for determining whether a CNS disorder in a pediatric patient is cancerous, wherein the composition comprises an agent capable of binding IL17A, an agent capable of binding IL1RA, and an agent capable of binding IP10.

In another aspect, the invention includes a composition useful for determining whether a CNS disorder in a pediatric patient is cancerous, wherein the composition comprises an agent capable of binding IL17A, an agent capable of binding IL1RA, an agent capable of binding IP10, and an agent capable of binding MDC.

In various embodiments of the above aspects or any other aspect of the invention delineated herein, the sample is cerebrospinal fluid (CSF).

In certain embodiments, the cytokine levels are measured using a technology selected from the group consisting of the Luminex FlexMPA 3D technology, microarray, sequencing, ELISA, and qPCR.

In certain embodiments, the agent is selected from the group consisting of an antibody, a probe, and a nucleotide sequence. In certain embodiments, the agent is bound to a microarray. In certain embodiments, the agent is fluorescently labeled.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1B depict a heat map and dendrogram based on agglomerative hierarchical clustering (AHC). FIG. 1A is a heat map visually depicting the cytokine levels shown on the right. The heat map was created in conjunction with agglomerative hierarchical clustering (AHC). The three classes shown in the AHC are reflected here. Vertical, dashed lines have been drawn to aid in this separation of classes. C=control; L=CNS B-cell lymphoma; IF=infection, T=tumor (high grade gliomas), A=autoimmune; MS=multiple sclerosis. FIG. 1B is an AHC based dendrogram that includes all 43 cases and shows separation of CNS diseases into three major classes demonstrating a trend toward clustering of disease types based on overall cytokine profiles.

FIG. 2 is a discriminant analysis observation plot. According to discriminant analysis, 100% of the 43 cases were assigned appropriately to their respective disease groups (infection, autoimmune, tumor, DM, control).

FIGS. 3A-3I illustrate results from Mann-Whitney analyses for specific cytokines: IP-10/CXCL10 (FIG. 3A, FIG. 3C, FIG. 3D), MDC/CCL22 (FIG. 3B), IL-7 (FIG. 3E), IL-8 (FIG. 3F), GRO/CXCL1 (FIG. 3G), VEGF (FIG. 3H), and PDGF-AA (FIG. 3I). Each graph shows the cytokine level distribution for the respective disease groups. In each graph, horizontal lines with an asterisk (*) indicate the presence of statistically significant differences between groups for the given cytokine. The number of asterisks corresponds to the calculated p-value (*=p<0.05, **=p<0.01, ***=p<0.005, ****=p<0.001).

FIGS. 4A-4F show ROC curves for IP-10/CXCL10 (FIG. 4A), MDC/CCL22 (FIG. 4B), IL-7 (FIG. 4C), IL-8 (FIG. 4D), GRO/CXCL1 (FIG. 4E), and PDGF-AA (FIG. 4F). The title of each graph includes the groups that were compared, as well as the respective AUC.

FIG. 5 illustrates an algorithm for diagnosis of CNS diseases using a selective cytokine panel. The cytokines included in the algorithm include IP-10/CXCL10, MDC/CCL22, IL-8, GRO/CXCL1, and PDGF-AA. Potential “cut-off” values for interpretation are presented here. Sensitivity, specificity, and likelihood ratios (if available) corresponding to the cut-off values have been generated from the ROC analyses and are also featured.

FIG. 6 is a Principal Component Analysis plot using principal components (PC) 1, 2 and 3. The PCA is based on the following cytokines: EGF, MDC/CCL22, PDGF-AA, Fractalkine/CX3CL1, IFN-γ GRO/CXCL1, IL-15, IL-2, IL-7, IL-8, IL-9, IP-10/CXCL10, TGF-α, IL12-p40, IL12-p70, IL13, IL-1β, and TNF-β.

FIG. 7 illustrates a linear discriminant analysis showing separation of CNS disease classes based on cytokine levels in adult patients. Spheres indicate the disease type for each individual sample: Autoimmune, infection, controls, lymphoma, and solid tumor.

FIG. 8 illustrates an agglomerative hierarchical clustering heat map, visually showing levels of cytokine in the various CNS disease classes in samples collected from adult patients.

FIG. 9 is a table illustrating the statistical differences between comparisons of groups, for example C-A is controls vs autoimmune, I-L is infections vs lymphoma.

FIG. 10 is a graph illustrating the use of Random Forest Machine Learning to identify cytokines in adult samples that are most informative in identifying CNS disease state. This analysis identified the following informative cytokines: MIP1A, GRO, IL-5, IL-10, MDC, and IL6.

FIG. 11 is a series of graphs illustrating the statistical differences between the different disease classes in adults showing the informative cytokines.

FIG. 12 illustrates a decision tree utilizing the informative cytokines identified using the Random Forest Machine Learning. This decision tree is more effective than the analysis of white blood cell count, glucose levels and protein levels, studies that are currently routinely done on CSF samples.

FIG. 13A-13B illustrate Mann-Whitney test analysis of the levels of IP-10/CXCL10 (FIG. 13A) and MDC (FIG. 13B) in CSF of pediatric patients using ELISA as a readout. CSF IP-10/CXCL10 levels are statistically greater in both CNS bacterial and viral infections compared to CNS tumors and CNS degenerative disorders and non-CNS disorders in pediatric patients. P-values represent the significance for the comparisons indicated by the bars.

FIG. 14 illustrates a Kruskal-Wallis and post-hoc Mann-Whitney analysis of cytokine expression between disease classes. Statistical analysis was used to determine significant differences between disease classes for all 41 cytokines. Comparisons are indicated in the title of each column; for example C-A is controls vs autoimmune, I-L is infections vs lymphoma. Comparisons highlighted in red are significant. 39 of 41 cytokines showed a significant difference among CNS disease classes.

FIG. 15 illustrates a heat map generated using agglomerative hierarchical clustering (AHC) of cytokine expression data from the adult patient population. AHC clustering was performed by first calculating the pairwise distance between all data points, followed by joining the data points that are the lease distant apart, and then repeatedly joining the next least distant pair of points until all points are joined. The relationships are represented by the dendrogram at the top of the chart.

FIG. 16 illustrates a linear discriminant analysis (LDA) of pediatric samples, which was used to investigate whether CSF cytokines could be used to cluster pediatric samples by CNS disease type.

FIG. 17 is a graph illustrating the use of unbiased random forest machine learning in order to identify cytokines with the highest ability to discriminate the CNS disease classes. Cytokines resulting in a Gini importance score over 0.04 were identified as being informative and included IL-17A, IL12p40, TNFα, IL1A, IP10, IL1RA, and MDC/CCL22.

FIG. 18 illustrates a heat map generated using the informative cytokines identified using the unbiased random forest machine learning demonstrated in FIG. 17. Expression analysis and hierarchical clustering demonstrated the relative levels of selected cytokines in the various disease states.

FIG. 19 is a chart illustrating an improved decision tree assembled using the cytokines identified by unbiased random forest machine learning as having the highest discriminatory power for pediatric samples.

FIG. 20 illustrates the use of the improved decision tree assembled using pediatric samples to suggest a diagnosis for a prospective patient. In this case, an 11 year old girl presented with acute flaccid myelitis. Tests for enterovirus were negative. Cytokine expression analysis in CSF was determined and applied to the decision tree. Expression levels of IL17A, IL-1RA, and IL-1A indicated that the disease state was likely autoimmune in nature.

FIG. 21 illustrates the use of the improved decision tree in the diagnosis of another pediatric patient. In this case a 7 year old girl presented with severe CNS symptoms that rapidly progressed to death. Standard CNS studies were negative. Cytokine expression in CSF, when applied to the decision tree algorithm indicated a possible autoimmune response as the likely cause of death.

FIG. 22 illustrates the use the of improved decision tree in the diagnosis of pediatric patient. A 7 year old girl presented with signs of meningitis. CNS cytokine expression analysis results were applied to the decision tree algorithm, which subsequently indicated that the condition was caused by a viral infection. Follow-up PCR analysis of CSF samples were positive for enterovirus, indicating the accuracy of the decision tree.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

When “an immunologically effective amount,” or “therapeutic amount” is indicated, the precise amount of the compositions of the present invention to be administered can be determined by a physician or researcher with consideration of individual differences in age, weight, tumor size, extent of infection or metastasis, and condition of the patient (subject).

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “limited toxicity” as used herein, refers to the peptides, polynucleotides, cells and/or antibodies of the invention manifesting a lack of substantially negative biological effects, anti-tumor effects, or substantially negative physiological symptoms toward a healthy cell, non-tumor cell, non-diseased cell, non-target cell or population of such cells either in vitro or in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a

“Parenteral” administration of an immunogenic composition includes, e.g., subcutaneous (s.c.), intravenous (i.v.), intramuscular (i.m.), or intrasternal injection, or infusion techniques.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “therapeutic” as used herein means a treatment and/or prophylaxis. A therapeutic effect is obtained by suppression, remission, or eradication of a disease state.

To “treat” a disease as the term is used herein, means to reduce the frequency or severity of at least one sign or symptom of a disease or disorder experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention includes compositions and methods for treating CNS disorders.

Pattern recognition receptors, such as toll-like receptors (TLRs), Nod-Like receptors (NLRs), and RIG-like receptors (RLRs), play a crucial role in responding to various insults and generating the innate response. The diversity and subsequent combinations of pattern recognition receptors situated in various cell types and subcellular compartments allows for detection of a wide array of cellular danger signals and ligands, resulting in the release of cytokines and induction of fairly customized inflammatory and anti-inflammatory responses. In view of this, the present invention involves the quantification of CSF cytokine levels and subsequent statistical analysis to investigate cytokine profiles of different CNS disease states and differentiate distinct neuropathologic processes.

The term “CNS disorder” or “neurological disorder” denotes any disorder which is present in the brain, spinal column, and related tissues, such as the meninges, which are responsive to an appropriate therapeutic agent. Among the various neurological disorders for which the method of the invention is effective are those which relate to a cell proliferative disease. The term “cell proliferative disease” embraces malignant as well as non-malignant cell populations which often appear morphologically to differ from the surrounding tissue. Thus, the cell proliferative disease may be due to a benign or a malignant tumor. In the latter instance, malignant tumors may be further characterized as being primary tumors or metastatic tumors, that is, tumors which have spread from systemic sites. Primary tumors can arise from glial cells (astrocytoma, oligodendroglioma, glioblastoma), ependymal cells (ependymoma) and supporting tissue (meningioma, schwannoma, papilloma of the choroid plexus). In children, tumors typically arise from more primitive cells (medulloblastoma, neuroblastoma, chordoma), whereas in adults astrocytoma and glioblastoma are the most common. However, the most common CNS tumors in general are metastatic, particularly those which infiltrate the leptomeninges. Tumors that commonly metastatically invade the meninges include non-Hodgkin's lymphoma, leukemia, melanoma, and adenocarcinoma of breast, lung, or gastrointestinal origin.

The term “meningitis” refers to inflammation of the meninges, or the protective membrane surrounding the brain and spinal or chord. This inflammation is typically caused by infection by either bacteria or viruses, however other causes including injury and cancer can result in similar inflammation. Differentiating between the different etiologies of meningitis is a key step in successful diagnosis and treatment and is often complicated by the similar signs and symptoms shared by both bacterial and viral meningitis. Bacterial meningitis is understood to be an inflammation of the meninges which may be caused by various types of bacteria, mainly three: meningococcus (Neisseria meningitidis), pneumococcus (Streptococcus pneumoniae) and Haemophilus influenzae type b. Viral meningitis is understood to be an inflammation of the meninges which can be caused by various viruses such as enteroviruses, including Echovirus, Coxsackie and, more rarely, viruses of the herpes group, such as herpes 1 and 2, Cytomegalovirus, Epstein-Barr virus, varicella-zona viruses, HHV6 virus and, more rarely, arboviruses.

The terms “autoimmune” or “demyelinating” disorders are commonly understood to be CNS disorders caused by antibodies or immune cells specific for autoantigens expressed by nerves or nervous tissue cells, with the resulting cytotoxicity causing the signs and symptoms of the disease. Examples of neurological autoimmune diseases affecting the CNS include multiple sclerosis (MS), chronic inflammatory demyelinating polyneuropathy (CIDP), Guillain-Barre-Syndrome (GBS), Fisher syndrome (FS), and Bickerstaff brainstem encephalitis (BBE). CNS autoimmune disorders frequently result in the “demyelination” of spinal or peripheral nerves secondary to CNS inflammation, wherein the myelin sheath deteriorates, thus disrupting or preventing neural conduction along the axon of the nerve.

### Samples

The methods of the present invention are based on the detection of the levels of cytokines in body fluid samples obtained from a subject suffering from a CNS disorder. The term “body fluid” refers to all fluids that are present in the body including but not limited to blood, lymph, urine, and cerebrospinal fluid (CSF). The blood sample may be a plasma sample or a serum sample. In a preferred embodiment of the present invention the cytokine levels are determined in a cerebrospinal fluid sample taken from the subject. The term “cerebrospinal fluid” or “CSF” is intended to include whole cerebrospinal fluid or derivatives of fractions thereof well known to those skilled in the art. Thus, a cerebrospinal fluid sample can include various fractionated forms of cerebrospinal fluid or can include various diluents added to facilitate storage or processing in a particular assay. Such diluents are well known to those skilled in the art and include various buffers, preservatives, and the like.

### Cytokine Measurement

The terms “cytokine” and “chemokine” refer to signaling proteins produced and secreted by cells to accomplish specific biological functions. Cytokines are frequently involved in autocrine, paracrine, and endocrine signaling as immunomodulating agents capable of controlling the balance between inflammation and immune tolerance, as well as between cellular and humoral immune responses. Chemokines are a type of cytokine that specifically results in the chemotaxis or the movement of immune cells between tissues of the body. Chemokine signaling is frequently pro-inflammatory, and is induced during an immune response in order to recruit immune cells to the site of infection.

Specific inflammation-related disorders, including those of the CNS, can be characterized and differentiated according to the types and amounts of cytokines and chemokines produced as a result of the pathology.

The present invention includes quantifying the levels of cytokines or chemokines in CSF samples taken from patents suspected of having a CNS disorder. Among the cytokines assayed include, but are not limited to, epidermal growth factor (EGF), fibroblast growth factor 2 (FGF2), eotaxin/CCL11, transforming growth factor alpha (TGF-α), granulocyte-colony stimulating factor (G-CSF), macrophage derived chemokine (MDC/CCL22), granulocyte macrophage colony-stimulating factor (GM-CSF), interferon-γ(IFN-γ), GRO/CXCL1, MCP3/CCL7, IL12p40, MCP-1/CCL-2, MIP1-a/CCL3, MIP1-b/(CCL4), tumor necrosis factor-a (TNF-a), tumor necrosis factor-β (TNF-β), IL-12p70, Fractalkine/CX3CL1, IL-la, IL-lb, IL-2, IL-4, IL-3, IL-5, IL-6, IL-7, IL-8, IL-9, IL-10, IL-13, IL-15, IL-17, IL-1Ra, IFN-a2, IP-1Q/CXCL10, sCD40L, FLT-3L, vascular endothelial growth factor (VEGF), platelet-derived growth factor AA (PDGF-AA), PDGF-AB/BB, RANTES, APRIL, BAFF/Blys, BRAK/CSCL14, CCL28, CXCL16, HMGB1, IFNλ3, IL-14, IL-19, IL-24, IL-28B, IL-32a, IL-34, IL-35, IL-36p, IL-37, IL-38, MW-4/PARC/CCL18, MPIF-1/CL23 and YKL40/CHI3L1, GCP2, HCC-1, 1-TAC, IL-11, IL-29, Lymphotactin, M-CSF, MIG, MIP-3a/CCL20, MIP-3p, NAP2, 6Ckine, BCA-1, CTACK, ENA-78, Eotaxin-2, Eotaxin-3, 1-309, IL-16, IL-20, IL-21, IL-23, IL-28A, IL-33, LIF, MCP-2, MCP-4, MIP-ld, CXCR4/SDF-1, CD117/SDF, TARC, TPO, TRAIL, and TSLP.

The levels of the specific cytokines and chemokines may be determined by any method known to those skilled in the art. Methods for assaying for a cytokine include but are not limited to Western blot, immunoprecipitation, immunoassay, immunohistochemistry, immunofluorescence and radioimmunoassay. Cytokine proteins analyzed may be localized intracellularly (most commonly an application of immunohistochemistry) or extracellularly. In certain embodiments, standard curves are generated for each cytokine, and median fluorescent intensities are transformed into concentrations by 5-point, non-linear regression.

The identification of cytokines and chemokines of the present invention may be accomplished using various suitable assays. A suitable assay may include one or more of a chemical assay, an enzyme assay, an immunoassay, mass spectrometry, chromatography, electrophoresis, a biosensor, an antibody microarray or any combination thereof. Most commonly if an immunoassay is used it may be an enzyme-linked immunosorbant assay (ELISA), a sandwich assay, a competitive or a non-competitive assay, a radioimmunoassay (RIA), a lateral flow immunoassay, a Western Blot, an electro-chemilumescent assay, a magnetic particle assay, an immunoassay using a biosensor, a bead-based array assay (e.g. Luminex, Milliplex or Bioplex), a multiplex aptamer-based assay (e.g. SOMAscan), an immunoprecipitation assay, an agglutination assay, a turbidity assay or a nephelometric assay.

Simultaneous analysis of different cytokines is provided by MILLIPLEX™ Human Cytokine/Chemokine Magnetic Bead Panel plates (MilliporeSigma Inc., Burlington, Mass., USA). In a preferred embodiment, the level of specific cytokines are detected by an immunoassay. As used herein, an “immunoassay” is an assay that utilizes an antibody to specifically bind to the antigen (i.e., the specific cytokine). The immunoassay is thus characterized by detection of specific binding of the specific cytokine to antibodies. Immunoassays for detecting specific cytokines may be either competitive or noncompetitive. Noncompetitive immunoassays are assays in which the amount of captured analyte (i.e., the specific cytokine) is directly measured. In competitive assays, the amount of analyte (i.e., the specific cytokine) present in the sample is measured indirectly by measuring the amount of an added (exogenous) analyte displaced (or competed away) from a capture agent (i.e., the antibody) by the analyte (i.e., the specific cytokine) present in the sample. In one competition assay, a known amount of the (exogenous) specific cytokine is added to the sample and the sample is then contacted with the antibody. The amount of added (exogenous) specific cytokine bound to the antibody is inversely proportional to the concentration of the specific cytokine in the sample before the specific cytokine is added. In one preferred “sandwich” assay, for example, the antibodies can be bound directly to a solid substrate where they are immobilized. These immobilized antibodies (capturing antibodies) then capture the cytokine peptide of interest present in the test sample. Other immunological methods include, but are not limited to, fluid or gel precipitation reactions, immunodiffusion (single or double), agglutination assays, immunoelectrophoresis, radioimmunoassays (RIA), enzyme-linked immunosorbent assays (ELISA), Western blots, liposome immunoassays (LIA; Monroe et al., 1986), complement-fixation assays, immunoradiometric assays, fluorescent immunoassays, protein A immunoassays, or immunoPCR. An overview of different immunoassays is given in Wild (2001), Ghindilis et al. (2002) and Price and Newman (1997).

Particularly advantageous to the present invention are systems in which the levels of the different cytokines, or the levels of the specific cytokines, possibly together with other biological markers, can be detected simultaneously. In this multi-parameter approach, antibodies may be coupled to microspheres or chips. An example of an immunoassay that provides for such simultaneous detection includes (but is not limited to) the xMap™ technology (Luminex FlexMAP3D, Austin, Tex., USA).

The cytokine-specific antibodies as discussed above can be used in the preparation of a diagnostic kit for use in the methods of treatment of the present invention. Accordingly, the present invention relates to a panel of cytokines as discussed above, for which antibodies that bind to and detect the cytokines can be used in the manufacture of a diagnostic kit for determining the nature of a CNS disorder and for the diagnosis of a subject suffering from a CNS disorder so that an appropriate treatment regimen can be administered.

### Methods of Diagnosing

Provided in the invention are methods of diagnosing a patient as having a particular type of Central Nervous System (CNS) disorder, for example by distinguishing the CNS disorder as being either bacterial, viral, autoimmune or cancerous.

In one aspect, the method comprises measuring cytokine levels in a sample from a patient and comparing the patient sample levels to a reference sample. When the levels of IP-10/CXCL10 and MDC/CCL22 are higher in the patient sample compared to the reference sample, the patient is diagnosed with an infectious, non-viral CNS disorder. When the level of IP-10/CXCL10 is higher in the patient sample compared to the reference sample, and the level of MDC/CCL22 is lower in in the patient sample compared to the reference sample, the patient is diagnosed with an infectious, viral CNS disorder. When the level of IP-10/CXCL10 is lower in the patient sample compared to the reference sample and the levels of IL-8 and GRO/CXCL1 are higher in the patient sample compared to the reference sample, the patient is diagnosed with a glioma. When the level of IP-10/CXCL10 is lower in the patient sample compared to the reference sample and the level of PDGF-AA is higher in the patient sample compared to the reference sample, the patient is diagnosed with a lymphoma. When the level of IP-10/CXCL10 is lower in the patient sample compared to the reference sample and the level of PDGF-AA is lower in the patient sample compared to the reference sample, the patient is diagnosed with an autoimmune or demyelinating disease.

In another aspect, the invention provides a method of diagnosing a patient as having a particular type of CNS disorder. The method comprises measuring the levels of IP-10/CXCL10, MDC/CCL22, IL-8, GRO/CXCL1, and PDGF-AA in a sample from the patient and carrying out the following steps: Step a): If the level of IP-10/CXCL10 is >2083 pg/ml, the CNS disorder is infectious, thus proceed to step b), and if the level of IP-10/CXCL10 is <2083 pg/ml, the CNS disorder is non-infectious thus proceed to step c). Step b): If the level of MDC/CCL22 is >390.3 pg/ml, the CNS disorder is diagnosed as a non-viral disorder, and if the MDC/CCL22 is <390.3 pg/ml, the CNS disorder is diagnosed as a viral disorder. Step c): If the level of IL-8 is >367.5 pg/ml and the level of GRO/CXCL1 is >190.6, the CNS disorder is diagnosed as a glioma and if the level of IL-8 is <367.5 pg/ml and the level of GRO/CXCL1 is <190.6, proceed to step d). Step d): If the level of PDGF-AA is >12.43, the CNS disorder is diagnosed as a lymphoma, and if the level of PDGF-AA is <12.43, the CNS disorder is diagnosed as an autoimmune disorder.

Also provided is a method of diagnosing a patient (e.g. a pediatric patient) as having a particular type of Central Nervous System (CNS) disorder. The method comprises measuring the levels of IL-6, MDC, MIP-1A, and IL-8 in a sample from the patient, comparing the patient sample levels to a first, second, third, fourth, fifth, sixth, and seventh reference sample, and carrying out the following steps: Step a): if the level of IL-6 is lower in the patient sample compared to the first reference sample, proceeding to step b), and if the level of IL-6 is higher in the patient sample compared to the first reference sample, proceeding to step c). Step b): if the level of MDC is higher in the patient sample compared to the second reference sample, the patients is diagnosed as having an autoimmune disorder. Step c): if the level of MDC is lower in the patient sample compared to the third reference sample, proceeding to step d) and if the level of MDC is higher in the patient sample compared to the third reference sample, proceeding to step e). Step d): if the level of MDC is higher in the patient sample compared to the fourth reference sample, the patient is diagnosed as having a lymphoma. Step e): if the level of MDC is lower in the patient sample compared to the fifth reference sample, and the level of MIP-1A is lower in the patient sample compared to the sixth reference sample, the patient is diagnosed as having cancer, and if the level of MDC is higher in the patient sample compared to the fifth reference sample, proceeding to step f). Step f): if the level of IL-8 is lower in the patient sample compared to the seventh reference sample, the patient is diagnosed as having a lymphoma, and if the level of IL-8 is higher in the patient sample compared to the seventh reference sample, the patient is diagnosed as having an infection.

In another aspect, the invention provides a method of diagnosing a pediatric patient as having a particular type of Central Nervous System (CNS) disorder. The method comprises measuring the levels of IL17A, IL1RA, IL1A, IP10, and MDC in a sample from the pediatric patient, and carrying out the following steps: Step a): If the level of IL17A is ≤12.51 pg/ml, the CNS disorder is diagnosed as a bacterial infection, and if the level of IL17A is ≤12.51 pg/ml, proceeding to step b). Step b): if the level of IL1RA is ≤37.64 pg/ml, proceed to step c), and if the level of IL1RA is >37.64 pg/ml, proceed to step d). Step c): if the level of IL1A is ≤8.02 pg/ml, the CNS disorder is diagnosed as an autoimmune disorder. Step d): if the level of IP10 is ≤4328.29 pg/ml, proceed to step e), and if the level of IP10 is >4328.29 pg/ml, proceeding to step f). Step e): if the level of IL1RA is ≤54.43 pg/ml the CNS disorder is diagnosed as an autoimmune disorder, and if the level of IL1RA is >54.43 pg/ml the CNS disorder is diagnosed as a cancer. Step f): if the level of MDC is ≤18.23 pg/ml, then the CNS disorder is diagnosed as viral, and if the level of MDC is >18.23 pg/ml, then the CNS disorder is diagnosed as a cancer.

Also provided is a method of diagnosing a patient (e.g. an adult patient) as having a particular type of Central Nervous System (CNS) disorder. The method comprises measuring the levels of IL-6, MDC, MIP-1A, and IL-8 in a sample from the patient, comparing the patient sample levels to a first, second, third, fourth, fifth, sixth, and seventh reference sample, and carrying out the following steps: Step a): if the level of IL-6 is higher in the patient sample compared to the first reference sample, proceeding to step b), and if the level of IL-6 is lower in the patient sample compared to the first reference sample, proceeding to step c). Step b): if the level of MDC is lower in the patient sample compared to the second reference sample, the patients is diagnosed as having an autoimmune disorder. Step c): if the level of MDC is higher in the patient sample compared to the third reference sample, proceeding to step d) and if the level of MDC is lower in the patient sample compared to the third reference sample, proceeding to step e). Step d): if the level of MDC is higher in the patient sample compared to the fourth reference sample, the patient is diagnosed as having a lymphoma. Step e): if the level of MDC is lower in the patient sample compared to the fifth reference sample, and the level of MIP-1A is lower in the patient sample compared to the sixth reference sample, the patient is diagnosed as having cancer, and if the level of MDC is higher in the patient sample compared to the fifth reference sample, proceeding to step f). Step f): if the level of IL-8 is lower in the patient sample compared to the seventh reference sample, the patient is diagnosed as having a lymphoma, and if the level of IL-8 is higher in the patient sample compared to the seventh reference sample, the patient is diagnosed as having an infection.

A reference sample can be any type of sample with a known concentration of one or more cytokines. For example, a reference sample can be a CSF sample with known concentrations of a particular cytokine or cytokines (e.g. IL-6, MDC, MIP-1A, and IL-8). The reference sample may be a sample from a patient with a particular disease or disorder (e.g. a bacterial infection, a viral infection, an autoimmune disease, or cancer). The reference sample can also be a standard curve, which comprises known quantities of a particular cytokine, or a sample containing multiple standard curves of multiple cytokines.

Identifying the CNS disease class causing the patient's symptoms can facilitate initiation of proper therapy and further testing if needed.

In certain embodiments, the sample is cerebrospinal fluid (CSF). In certain embodiments, the cytokine levels are measured using a technology selected from the group consisting of the Luminex FlexMPA 3D technology, microarray, sequencing, ELISA, and qPCR.

One or more statistical analyses may be carried out with the methods disclosed herein. Statistical analyses can include, but are not limited to, Kruskal-Wallis and post-hoc Mann-Whitney tests, ANOVA, and random forest.

### Compositions

The invention also provides compositions for use in diagnosing patients with a particular type of CNS disorder, for example distinguishing the CNS disorder as being infectious (e.g. related to a viral or bacterial infection), autoimmune, or cancerous (e.g. related to a lymphoma or glioma).

One aspect of the invention provides a composition useful for determining whether a CNS disorder in a patient is infectious. The composition comprises an agent capable of binding IP-10/CXCL10, and an agent capable of binding MDC/CCL22.

Another aspect of the invention provides a composition useful for determining whether a CNS disorder in a patient is a lymphoma, a glioma, or an autoimmune disorder. The composition comprises an agent capable of binding IP-10/CXCL10, an agent capable of binding IL-8, an agent capable of binding GRO/CXCL1, and an agent capable of binding PDGF-AA.

Yet another aspect of the invention provides a composition useful for determining whether a CNS disorder in a pediatric patient is an autoimmune disorder. The composition comprises an agent capable of binding IL17A, an agent capable of binding IL1RA, and an agent capable of binding IL1A.

Also provided in the invention is a composition useful for determining whether a CNS disorder in a pediatric patient is an autoimmune disorder. The composition comprises an agent capable of binding IL17A, an agent capable of binding IL1RA, and an agent capable of binding IP10.

Also provided is a composition useful for determining whether a CNS disorder in a pediatric patient is bacterial. The composition comprises an array comprising an agent capable of binding IL17A and an agent capable of binding IL1RA.

Also provided is a composition useful for determining whether a CNS disorder in a pediatric patient is viral. The composition comprises an array comprising an agent capable of binding IL17A, an agent capable of binding IL1RA, an agent capable of binding IP10, and an agent capable of binding MDC.

Also provided is a composition useful for determining whether a CNS disorder in a pediatric patient is cancerous. The composition comprises an agent capable of binding IL17A, an agent capable of binding IL1RA, and an agent capable of binding IP10.

Also provided is a composition useful for determining whether a CNS disorder in a pediatric patient is cancerous. The composition comprises an agent capable of binding IL17A, an agent capable of binding IL1RA, an agent capable of binding IP10, and an agent capable of binding MDC.

Agents that can be used include, but are not limited to, an antibody, a probe, and a nucleotide sequence.

In certain embodiments, the agent is bound to a microarray. In certain embodiments, the agent is fluorescently labeled.

In certain embodiments, the composition may be part of a kit. Kits of the invention may be used for practicing the invention methods, as described herein. Kits for practicing the invention methods may include any of the compositions disclosed herein. Additional reagents that are required or desired in the protocol to be practiced with the kit components may be present. Examples of additional reagents can include, but are not limited to: standards, carriers, PCR amplification reagents (e.g., nucleotides, buffers, cations, etc.), and the like. The kit components may be present in separate containers, or one or more of the components may be present in the same container, where the containers may be storage containers and/or containers that are employed during the assay for which the kit is designed.

In addition to the above components, the kit may further include instructions for practicing the methods described herein. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of instructions may include a computer readable medium, e.g., diskette, CD, etc., on which the information has been recorded. Yet another form of instructions may include a website address which may be used via the internet to access the information at a removed site. Any convenient means may be present in the kits.

### Methods of Treatment

The present invention includes methods for treating Central Nervous System (CNS) disorders. In one aspect, the method comprises measuring cytokine levels from a sample from a patient and comparing the patient sample levels to a reference sample. When the levels of IP-10/CXCL10 and MDC/CCL22 are higher in the patient sample compared to the reference sample, the CNS disorder is an infectious, non-viral disorder, and an anti-bacterial and/or anti-fungal and/or anti-parasitic treatment is administered to the patient. When the level of IP-10/CXCL10 is higher in the patient sample compared to the reference sample, and the level of MDC/CCL22 is lower in in the patient sample compared to the reference sample, the CNS disorder is an infectious, viral disorder, and an anti-viral treatment is administered to the patient. When the level of IP-10/CXCL10 is lower in the patient sample compared to the reference sample and the levels of IL-8 and GRO/CXCL1 are higher in the patient sample compared to the reference sample, the CNS disorder is a glioma, and an treatment for gliomas is administered to the patient. When the level of IP-10/CXCL10 is lower in the patient sample compared to the reference sample and the level of PDGF-AA is higher in the patient sample compared to the reference sample, the CNS disorder is a lymphoma, and an treatment for lymphomas is administered to the patient.

In another aspect, the invention includes a method of treating a CNS disorder comprising measuring the levels of IP-10/CXCL10, MDC/CCL22, IL-8, GRO/CXCL1, and PDGF-AA in a sample from the patient, and carrying out the following steps: Step a): if the level of IP-10/CXCL10 is >2083 pg/ml, the CNS disorder is infectious, thus proceed to step b). If the level of IP-10/CXCL10 is <2083 pg/ml, the CNS disorder is non-infectious, thus proceed to step c). Step b): if the level of MDC/CCL22 is >390.3 pg/ml, the CNS disorder is a non-viral disorder, thus an anti-bacterial and/or anti-fungal and/or anti-parasitic treatment is administered to the patient. If the MDC/CCL22 is <390.3 pg/ml, the CNS disorder is a viral disorder, thus an anti-viral treatment is administered to the patient. Step c): if the level of IL-8 is >367.5 pg/ml and the level of GRO/CXCL1 is >190.6, the CNS disorder is a glioma, thus a treatment for gliomas is administered to the patient. If the level of IL-8 is <367.5 pg/ml and the level of GRO/CXCL1 is <190.6, proceed to step d). Step d) if the level of PDGF-AA is >12.43, the CNS disorder is a lymphoma, thus a treatment for lymphomas is administered to the patient. If the level of PDGF-AA is <12.43, the CNS disorder is an autoimmune disorder, thus a treatment for an autoimmune disorder is administered to the patient.

Also provided is a method of treating a Central Nervous System (CNS) disorder in a pediatric patient in need thereof. The method comprises measuring the levels of IL17A, IL1RA, IL1A, IP10, and MDC in a sample from the pediatric patient, comparing the patient sample levels to a reference sample, and carrying out the following steps: Step a): if the level of IL17A is higher in the patient sample compared to the reference sample, the CNS disorder is a bacterial infection and an anti-bacterial treatment is administered to the patient, and wherein when the level of IL17A is lower in the patient sample compared to the reference sample, proceeding to step b). Step b): if the level of IL1RA is lower in the patient sample compared to the reference sample, proceeding to step c), and if the level of IL1RA is higher in the patient sample compared to the reference sample, proceeding to step d). Step c): if the level of IL1A is lower in the patient sample compared to the reference sample, the CNS disorder is an autoimmune disorder and a treatment for an autoimmune disorder is administered to the patient. Step d) if the level of IP10 is lower in the patient sample compared to the reference sample, proceeding to step e), and if the level of IP10 is higher in the patient sample compared to the reference sample, proceeding to step f). Step e): if the level of IL1RA is lower in the patient sample compared to a second reference sample, the CNS disorder is an autoimmune disorder and a treatment for an autoimmune disorder is administered to the patient, and if the level of IL1RA is higher in the patient sample compared to a second reference sample, the CNS disorder is a cancer and a treatment for cancer is administered to the patient. Step f): if the level of MDC is lower in the patient sample compared to the reference sample, then the CNS disorder is viral and an anti-viral treatment is administered to the patient, and if the level of MDC higher in the patient sample compared to the reference sample, then the CNS disorder is a cancer and a treatment for cancer is administered to the patient.

Also provided is a method of treating a CNS disorder in a pediatric patient in need thereof. The method comprises measuring the levels of IL17A, IL1RA, IL1A, IP10, and MDC in a sample from the pediatric patient, and carrying out the following steps: Step a) if the level of IL17A is >12.51 pg/ml, the CNS disorder is a bacterial infection and an anti-bacterial treatment is administered to the patient. If the level of IL17A is ≤12.51 pg/ml, proceed to step b). Step b): if the level of IL1RA is ≤37.64 pg/ml, proceed to step c), and if the level of IL1RA is >37.64 pg/ml, proceeding to step d). Step c) if the level of IL1A is ≤8.02 pg/ml, the CNS disorder is an autoimmune disorder and a treatment for an autoimmune disorder is administered to the patient. Step d): if the level of IP10 is ≤4328.29 pg/ml, proceed to step e), and if the level of IP10 is >4328.29 pg/ml, proceed to step f. Step e): if the level of IL1RA is ≤54.43 pg/ml the CNS disorder is an autoimmune disorder and a treatment for an autoimmune disorder is administered to the patient. If the level of IL1RA is >54.43 pg/ml the CNS disorder is a cancer and a treatment for cancer is administered to the patient. Step f): if the level of MDC is ≤18.23 pg/ml, then the CNS disorder is viral and an anti-viral treatment is administered to the patient. If the level of

MDC is >18.23 pg/ml, then the CNS disorder is a cancer and a treatment for cancer is administered to the patient.

In another aspect, the invention provides a method of treating a CNS disorder in a patient in need thereof. The method comprises measuring the levels of IL-6, MDC, MIP-1A, and IL-8 in a sample from the patient, comparing the patient sample levels to a first, second, third, fourth, fifth, sixth, and seventh reference sample, and carrying out the following steps: Step a): if the level of IL-6 is lower in the patient sample compared to the first reference sample, proceeding to step b), and if the level of IL-6 is higher in the patient sample compared to the first reference sample, proceeding to step c). Step b): if the level of MDC is higher in the patient sample compared to the second reference sample, the patients is diagnosed as having an autoimmune disorder. Step c): if the level of MDC is lower in the patient sample compared to the third reference sample, proceeding to step d) and if the level of MDC is higher in the patient sample compared to the third reference sample, proceeding to step e). Step d): if the level of MDC is higher in the patient sample compared to the fourth reference sample, the patient is diagnosed as having a lymphoma. Step e): if the level of MDC is lower in the patient sample compared to the fifth reference sample, and the level of MIP-1A is lower in the patient sample compared to the sixth reference sample, the patient is diagnosed as having cancer, and if the level of MDC is higher in the patient sample compared to the fifth reference sample, proceeding to step f). Step f): if the level of IL-8 is lower in the patient sample compared to the seventh reference sample, the patient is diagnosed as having a lymphoma, and if the level of IL-8 is higher in the patient sample compared to the seventh reference sample, the patient is diagnosed as having an infection. Based on the patient's diagnosis, a specific treatment is administered (e.g. if the patient is diagnosed as having cancer, a cancer treatment is administered to the patient).

In another aspect, the invention provides a method of treating a CNS disorder comprising measuring cytokine levels in a sample from a patient and comparing the patient sample levels to a reference sample, wherein when the level of IP-10/CXCL10 is lower in the patient sample compared to the reference sample, the CNS disorder is non-infectious, and when the level of IP-10/CXCL10 is higher in the patient sample compared to the reference sample, the CNS disorder is infectious, and a treatment is administered to the patient that treats the infection.

In certain embodiments, when the CNS disorder is infectious and the level of MDC/CCL22 is higher in the patient sample compared to the reference sample, the CNS disorder is a non-viral disorder, and an anti-bacterial and/or anti-fungal and/or anti-parasitic treatment is administered to the patient, and wherein when the level of MDC/CCL22 is lower in in the patient sample compared to the reference sample, the CNS disorder is a viral disorder, and an anti-viral treatment is administered to the patient.

In certain embodiments, when the CNS disorder is non-infectious, and the levels of IL-8 and GRO/CXCL1 are higher in the patient sample compared to the reference sample, the CNS disorder is a glioma, and a treatment for gliomas is administered to the patient, and when the level of IL-8 and GRO/CXCL1 are lower in the patient sample compared to the reference sample, the CNS disorder is an autoimmune disorder or a lymphoma, and a treatment for an autoimmune disorder or a lymphoma is administered to the patient.

In certain embodiments, when the CNS disorder is an autoimmune disorder or a lymphoma, and when the level PDGF-AA is higher in the patient sample compared to the reference sample, the CNS disorder is a lymphoma, and an treatment for lymphomas is administered to the patient, and when the level PDGF-AA is lower in the patient sample compared to the reference sample, the CNS disorder is an autoimmune disorder and a treatment for an autoimmune disorder is administered to the patient .

In another aspect, the invention provides a method of treating a CNS disorder in a pediatric patient in need thereof. The method comprises measuring cytokine levels in a sample from the pediatric patient, and comparing the patient sample levels to a reference sample, wherein when the level of IL17A is lower in the patient sample compared to the reference sample, the CNS disorder is not a bacterial infection, and wherein when the level of IL17A is higher in the patient sample compared to the reference sample, the CNS disorder is a bacterial infection and an anti-bacterial treatment is administered to the patient.

In certain embodiments, when the CNS disorder is not a bacterial infection, and the level of IL1RA is higher in the patient sample compared to the reference sample, and the level of IL1A is higher in the patient sample compared to the reference sample, the CNS disorder is an autoimmune disorder and a treatment for an autoimmune disorder is administered to the patient.

In certain embodiments, when the CNS disorder is not a bacterial infection, and the level of IL1RA is lower in the patient sample compared to the reference sample, and the level of IP10 is higher in the patient sample compared to the reference sample, and the level of IL1RA is higher in the patient sample compared to a second reference sample, the CNS disorder is an autoimmune disorder and a treatment for an autoimmune disorder is administered to the patient.

In certain embodiments, when the CNS disorder is not a bacterial infection, and the level of IL1RA is lower in the patient sample compared to the reference sample, and the level of IP10 is higher in the patient sample compared to the reference sample, and the level of IL1RA is lower in the patient sample compared to a second reference sample, the CNS disorder is a cancer and a treatment for cancer is administered to the patient.

In certain embodiments, when the CNS disorder is not a bacterial infection, and the level of IL1RA is lower in the patient sample compared to the reference sample, and the level of IP10 is lower in the patient sample compared to the reference sample, and the level of MDC is higher in the patient sample compared to the reference sample, the CNS disorder is viral and an anti-viral treatment is administered to the patient.

In certain embodiments, when the CNS disorder is not a bacterial infection, and the level of IL1RA is lower in the patient sample compared to the reference sample, and the level of IP10 is lower in the patient sample compared to the reference sample, and the level of MDC is lower in the patient sample compared to the reference sample, the CNS disorder is a cancer and a treatment for cancer is administered to the patient. In another aspect, the invention includes a method of treating a Central Nervous System (CNS) disorder comprising measuring cytokine levels in a sample from a patient and comparing the patient sample levels to a reference sample, wherein when the level of IL-6 is lower in the patient sample compared to the reference sample, and the level of MDC is higher in the patient sample compared to the reference sample, the CNS disorder is an autoimmune disorder and a treatment for an autoimmune disorder is administered to the patient.

In another aspect, the invention includes a method of treating a Central Nervous System (CNS) disorder comprising measuring cytokine levels in a sample from a patient and comparing the patient sample levels to a first, second, and third reference sample. When the level of IL-6 is higher in the patient sample compared to the first reference sample, and the level of MDC is lower in the patient sample compared to the second reference sample and compared to the third reference sample, the CNS disorder is an autoimmune disorder and a treatment for an autoimmune disorder is administered to the patient.

In another aspect, the invention includes a method of treating a Central Nervous System (CNS) disorder comprising measuring cytokine levels in a sample from a patient and comparing the patient sample levels to a first, second, and third reference sample. When the level of IL-6 is higher in the patient sample compared to the first reference sample, and the level of MDC is lower in the patient sample compared to the second reference sample but higher compared to the third reference sample, the CNS disorder is a lymphoma and a treatment for lymphoma is administered to the patient.

In another aspect, the invention includes a method of treating a Central Nervous System (CNS) disorder comprising measuring cytokine levels in a sample from a patient and comparing the patient sample levels to a first, second, third, and fourth reference sample. When the level of IL-6 is higher in the patient sample compared to the first reference sample, and the level of MDC is higher in the patient sample compared to the second reference sample but lower compared to the third reference sample, and the level of MIP-1A is higher in the patient sample compared to the fourth reference sample, the CNS disorder is a cancer and a treatment for cancer is administered to the patient.

In another aspect, the invention includes a method of treating a Central Nervous System (CNS) disorder comprising measuring cytokine levels in a sample from a patient and comparing the patient sample levels to a first, second, third, and fourth reference sample. When the level of IL-6 is higher in the patient sample compared to the first reference sample, and the level of MDC is higher in the patient sample compared to the second reference sample but lower compared to the third reference sample, and the level of MIP-1A is lower in the patient sample compared to the fourth reference sample, the CNS disorder is a cancer and a treatment for cancer is administered to the patient.

In another aspect, the invention includes a method of treating a Central Nervous System (CNS) disorder comprising measuring cytokine levels in a sample from a patient and comparing the patient sample levels to a first, second, third, and fourth reference sample. When the level of IL-6 is higher in the patient sample compared to the first reference sample, and the level of MDC is higher in the patient sample compared to the second reference sample and the third reference sample, and the level of IL-8 is lower in the patient sample compared to the fourth reference sample the CNS disorder is a lymphoma and a treatment for lymphoma is administered to the patient.

In another aspect, the invention includes a method of treating a Central Nervous System (CNS) disorder comprising measuring cytokine levels in a sample from a patient and comparing the patient sample levels to a first, second, third, and fourth reference sample. When the level of IL-6 is higher in the patient sample compared to the first reference sample, and the level of MDC is higher in the patient sample compared to the second reference sample and the third reference sample, and the level of IL-8 is higher in the patient sample compared to the fourth reference sample, the CNS disorder is infectious and an anti-bacterial and/or anti-fungal and/or anti-parasitic treatment and/or anti-viral treatment is administered to the patient.

As used herein, a “reference sample” can be any type of sample with a known concentration of one or more cytokines. For example, a reference sample can be a CSF sample with known concentrations of a particular cytokine or cytokines (e.g. IL-6, MDC, MIP-1A, and IL-8). The reference sample may be a sample from a patient with a particular disease or disorder (e.g. a bacterial infection, a viral infection, an autoimmune disease, or cancer). The reference sample can also be a standard curve, which comprises known quantities of a particular cytokine, or a sample containing multiple standard curves of multiple cytokines.

In certain embodiments, the sample is cerebrospinal fluid (CSF). In certain embodiments, the cytokine levels are measured using a technology selected from the group consisting of the Luminex FlexMPA 3D technology, microarray, sequencing, ELISA, and qPCR.

One or more statistical analyses may be carried out with the methods disclosed herein. Statistical analyses can include, but are not limited to, Kruskal-Wallis and post-hoc Mann-Whitney tests, ANOVA, and random forest.

Treatments of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials. The treatment may be carried out in any convenient manner known to those of skill in the art. The treatment may be administered to a subject by aerosol inhalation, injection, ingestion, transfusion, implantation or transplantation. The treatment may be administered to a patient transarterially, subcutaneously, intradermally, intratumorally, intranodally, intramedullary, intramuscularly, by intravenous (i. v.) injection, or intraperitoneally. In other instances, the treatment is injected directly into a site of inflammation in the subject, a local disease site in the subject, a lymph node, an organ, a tumor, and the like.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in these experiments are now described.

Populations: The CSF samples analyzed were de-identified and no longer needed for clinical analysis. The samples were originally obtained by lumbar puncture as part of a clinical work-up of patients prior to specimen de-identification, and all samples were obtained with appropriate consent by the clinical team during the clinical evaluation. Personal data of patients was protected at all times. Criteria for inclusion were patients at TJUH age 2 to 80 years of age with CNS disease. CSF laboratory studies, such as white blood cell (WBC) count, CSF glucose concentration, and CSF protein levels, were a component of the patient's clinical evaluation. Samples selected for cytokine analysis included control patients negative for neuro-inflammatory processes, patients with CNS infections, patients with malignant glial (astrocytic) neoplasms, patients with autoimmune and demyelinating disease (autoimmune/DM), and patients with B-cell lymphoma involving the CNS.

Cytokine analysis: All CSF samples (n=43) were analyzed using the human Cytokine/Chemokine Magnetic Bead Panel Millipore plates on a Luminex FlexMPA 3D for the following analytes: epidermal growth factor (EGF), fibroblast growth factor 2 (FGF2), eotaxin/CCL11, transforming growth factor alpha (TGF-α), granulocyte-colony stimulating factor (G-CSF), macrophage derived chemokine (MDC/CCL22), granulocyte macrophage colony-stimulating factor (GM-CSF), interferon-γ (IFN-γ), GRO/CXCL1, MCP3/CCL7, IL12p40, MCP-1/CCL-2, MIP1-α/CCL3, MIP1-β/C CL4, tumor necrosis factor-α (TNF-α), tumor necrosis factor-β (TNF-β), IL-12p70, Fractalkine/CX3CL1, IL-1α, IL-1β, IL-2, IL-4, IL-3, IL-5, IL-6, IL-7, IL-8, IL-9, IL-10, IL-13, IL-15,IL-17α, IL-1Ra, IFN-α2, IP-10/CXCL10, sCD40L, FLT-3L, vascular endothelial growth factor (VEGF), platelet-derived growth factor AA (PDGF-AA), PDGF-AB/BB, and RANTES. Samples were analyzed in duplicate by a FlexMAP 3D (Luminex). Standard curves were generated for each cytokine, and median fluorescent intensities were transformed into concentrations by 5-point, non-linear regression. Data was exported to a Microsoft Excel file.

Statistical analyses: Statistical analyses including agglomerative hierarchical analysis, discriminant analysis (DA), and principal component analysis (PCA) were performed using the XLStat statistics program. These methods help assess the relationships of cytokine profiles based in the innate immune response reflected by CSF cytokine levels. DA was performed to determine informative cytokines. Informative cytokines were then used for generation of three-dimensional PCA plots to identify disease-type clustering as a function of informative cytokine levels. The Mann-Whitney test for univariate, non-parametric analysis using the Prism GraphPad Statistics Program was applied for comparison of cytokines among the disease groups (controls, infections, gliomas, autoimmune/DM, lymphomas). Receiver operator characteristic (ROC) analysis was performed using Prism GraphPad Statistics Program.

The results of the experiments are now described.

### Example 1

### Correlation of CSF Cytokine Expression and CNS Disease State

CSF samples from 43 patients were collected, spanning a wide range of CNS diseases: various infections (viral, bacterial, fungal and protozoan), autoimmune and demyelinating diseases, lymphomas, and gliomas. A variety of different pathogens were included in the infectious group to reproduce the common clinical scenario in which a range of pathogens are in the differential diagnosis to exclude infection. The control samples were from non-infectious cases with a thorough, negative clinical work-up, and included diagnoses such as idiopathic intracranial hypertension, headache, and hydrocephalus. The diagnosis, patient age, and sex for each case are presented in Table 1. The median age of the control group was 50 years. The cases included seven controls, 15 infectious cases (three fungal, seven viral, five bacterial, and one protozoan), three malignant astrocytic glioma cases, 12 autoimmune/demyelinating cases, and six cases of B-cell lymphomas involving the CNS (four primary CNS lymphomas of diffuse large B-cell type and two systemic B-cell lymphomas involving the CNS).

TABLE 1
Disease Class, Specific Diagnosis, Age, and Sex for Cases (CSF samples)

| | | Age | |
|---|---|---|---|
| Disease Class | Diagnosis | (years) | Gender |
| Control | Headache | 50 | F |
| Control | Transient ischemic attack | 77 | F |
| Control | Idiopathic intracranial hypertension | 40 | F |
| Control | Headache | 55 | F |
| Control | Hydrocephalus/VP shunt | 51 | F |
| Control | Headache | 33 | F |
| Control | Hydrocephalus/VP Shunt | 2 | M |
| Infection | Cryptococcal meningitis/HIV | 54 | M |
| Infection | Enterovirus meningitis | 19 | M |
| Infection | JC virus/PML/HIV | 46 | M |
| Infection | HPeV encephalitis | 17 | F |
| Infection | Cryptoccal meningitis post heart transplant | 55 | M |
| Infection | WNV encephalitis | 33 | M |
| Infection | Lyme disease | 48 | M |
| Infection | Toxoplasmosis/status post chemotherapy | 73 | F |
| Infection | Cryptococcal meningitis/HIV | 42 | M |
| Infection | TB meningitis post adalimumab therapy | 30 | F |
| Infection | Staphylococcus epidermidis meningitis/HIV | 32 | M |
| Infection | Streptococcus mitis meningitis | 73 | M |
| Infection | Viral meningitis, not otherwise specificied | 20 | F |
| Infection | TB meningitis/HIV | 35 | F |
| Infection | JCV meningitis/immunosuppression for lupus | 52 | F |
| Glioma | Anaplastic astrocytoma | 77 | F |
| Glioma | Recurrent glioblastoma | 60 | M |
| Glioma | Recurrent glioblastoma | 50 | F |
| Autoimmune/DM | Autoimmune encephalopathy | 53 | F |
| Autoimmune/DM | Post-viral cerebellitis | 51 | F |
| Autoimmune/DM | Transverse myelitis | 80 | F |
| Autoimmune/DM | CNS vasculitis | 27 | F |
| Autoimmune/DM | Paraneoplastic cerebellar dysfunction | 55 | M |
| Autoimmune/DM | Acute disseminated encephalomyelitis | 39 | M |
| Autoimmune/DM | Acute disseminated encephalomyelitis | 80 | M |
| Autoimmune/DM | Anti-acetylcholine ganglionic neuronal receptor | 66 | F |
| | autoimmune encephalopathy | | |
| Autoimmune/DM | Multiple sclerosis | 29 | F |
| Autoimmune/DM | Multiple sclerosis | 30 | F |
| Autoimmune/DM | Multiple sclerosis | 46 | M |
| Autoimmune/DM | Tumefactive multiple sclerosis | 30 | F |
| Lymphoma | Primary CNS Lymphoma | 21 | M |
| Lymphoma | Primary CNS Lymphoma | 72 | M |
| Lymphoma | Primary CNS Lymphoma | 58 | F |
| Lymphoma | Primary CNS Lymphoma | 78 | M |
| Lymphoma | Systemic DLBCL involving CNS | 69 | F |
| Lymphoma | Systemic Burkitt lymphoma involving CNS | 37 | M |

The initial CSF parameters routinely measured in patients with suspected CNS disease include CSF WBC count (cells/μl), CSF protein concentration (mg/dl), and CSF glucose concentration (mg/dl). Summaries of these findings are shown in Table 2. Using Mann-Whitney tests of significance, it was found that the protein levels in the infection group and the glioma group were statistically higher than the protein levels in the control group. Other than these two statistical differences, with the numbers of samples available for analysis, there were no other significant differences between any of the CNS disease groups for any of the CNS parameters listed above.

TABLE 2
Patient Age and CSF WBC Count, Protein Concentration, and Glucose Data

| | | | | Autoimmune/ | |
|---|---|---|---|---|---|
| | Controls | Infections | Gliomas | Demyelinating | Lymphomas |
| n | 7 | 15 | 3 | 12 | 6 |
| Age, years | 50(2-77) | 39(17-73) | 60(50-77) | 51(27-88) | 64(21-78) |
| CSF | 2(0-2) | 29(0-511) | 7(0-263) | 9(0-119) | 10(0-25) |
| WBC(cells/μl) | | | | | |
| (normal = 0) | | | | | |
| CSF Protein | 33(16-47) | 70*(19-910) | 124*(50-133) | 47(14-183) | 47(25-105) |
| (mg/dl) (normal | | | | | |
| range = 15-55) | | | | | |
| CSF Glucose | 68(32-82) | 60(32-87) | 66(11-89) | 64(42-197) | 78(50-95) |
| (mg/dl) (normal | | | | | |
| range = 40-70) | | | | | |

Values for age, CSF WBC, CSF Protein, and CSF Glucose are medians (minimum-maximum).
*The CSF protein levels in the infection group and the glioma group were statistically different from the CSF proteins levels in the control group (p = 0.0143 and p = 0.0163 respectively).

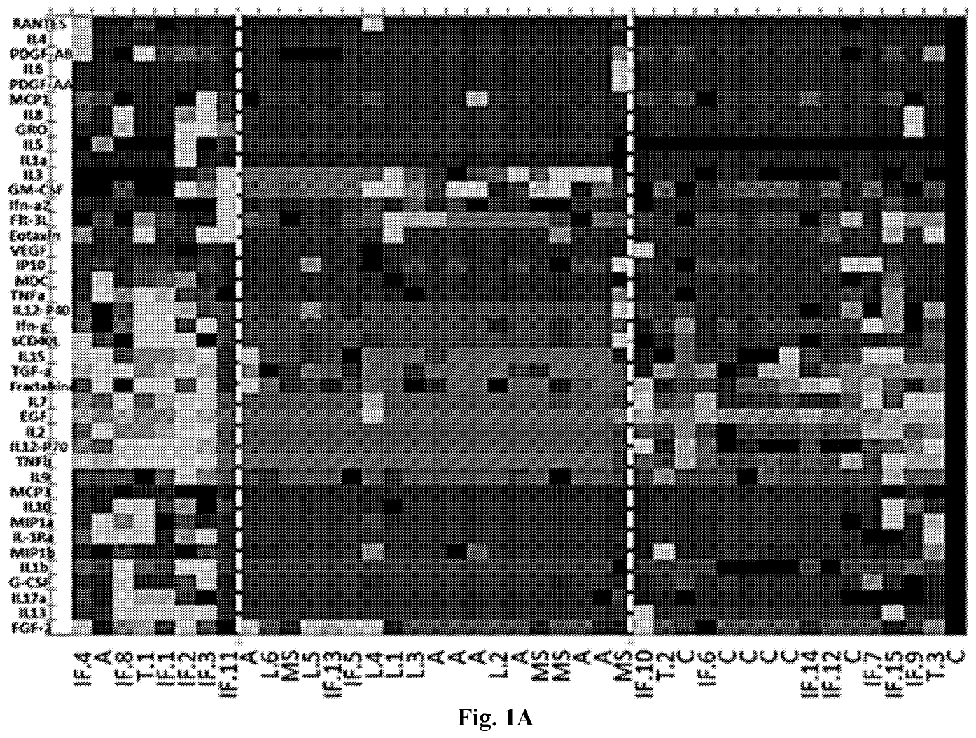

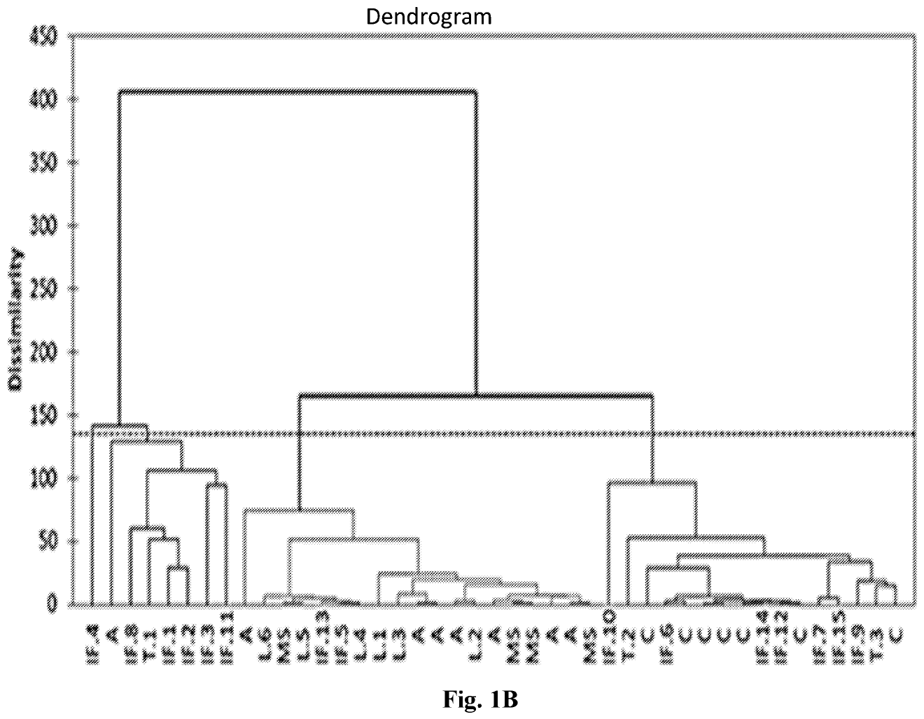

A heat map and dendrogram were generated using all 43 patient samples with the measured levels from 41 cytokines: epidermal growth factor (EGF), fibroblast growth factor 2 (FGF2), eotaxin/CCL11, transforming growth factor alpha (TGF-α), granulocyte-colony stimulating factor (G-CSF), macrophage derived chemokine (MDC/CCL22), granulocyte macrophage colony-stimulating factor (GM-CSF), interferon-γ (IFN-γ), GRO/CXCL1, MCP3/CCL7, IL12p40, MCP-1/CCL-2, MIP1-α/CCL3, MIP1-β/CCL4, tumor necrosis factor-α (TNF-α), tumor necrosis factor-β (TNF-β), IL-12p70, fractalkine/CX3CL1, IL-1α, IL-1β, IL-2, IL-4, IL-3, IL-5, IL-6, IL-7, IL-8, IL-9, IL-10, IL-13, IL-15, IL-17α, IL-1Ra, IFN-α2, IP-10/CXCL10, sCD40L, FLT-3L, vascular endothelial growth factor (VEGF), platelet-derived growth factor AA (PDGF-AA), PDGF-AB/BB, and RANTES (FIG. 1). These analytical tools help to visualize large sets of data to assess for overarching trends and patterns. The heat map (FIG. 1A) displays all 43 cases with the corresponding CSF cytokine levels represented by the color scale. Each column in the graph represents a case. By overall cytokine levels, the cases separate into major classes based on three predominant cytokine profiles. Two vertical lines are superimposed on the heat map to further illustrate these classes. These data reveal that profiles composed of levels (pg/ml) of multiple cytokines in the CSF display relative sterotyped responses to the various disease processes that occur in the CNS. These profiles resulting from these sterotypical disease specific responses can be used to help identify the disease class that is harming the patients CNS. How the mathematical/statistical analysis of the CSF cytokine levels contributes to identifying CNS disease class is described below.

The class distinctions demonstrated by the heat map correspond directly to the classes depicted in the dendrogram (FIG. 1B). Agglomerative hierarchical clustering (AHC) was generated from data expressed by the heat map. AHC is based on Ward's method calculation to minimize the variance within each cluster. Successive clustering progresses in a “bottom-up” approach in order to create homogenous classes or clusters of diseases arranged graphically in the form of a dendrogram.

The dendrogram (FIG. 1B) includes all 43 cases and shows separation of these cases into three broad classes. One class contained the vast majority of the autoimmune/DM cases and all lymphoma cases. The few CNS infections included in this class were in severely immunosuppressed patients with immunosuppression-related infections: two cases of JC virus progressive multifocal leukoencephalopathy (PML) in a patient with a heart transplant and in a patient with human immunodeficiency virus (HIV), and one case of neurotuberculosis in a patient with HIV. On the heat map, this class shows an overall subdued cytokine pattern, displaying the least relative increases in cytokine levels (this is indicated by the predominance of red colors on the graph).

The second class on the dendrogram includes WHO grade IV malignant astrocytic neoplasms; CNS fungal infections (cerebromeningeal cryptococcus infection); CNS viral infections (West Nile virus and human parechovirus meningitis); CNS protozoan infections (Toxoplasmosis); and the control cases. On the heat map, this class generally showed intermediate cytokine levels, ranging between the levels observed in the first class and the third class.

The third class corresponds to diseases with the most pronounced increase of cytokine levels shown on the heat map, which included eight cases: a fatal case of rotuberculosis in a patient treated with adalimumab; enterovirus (EV) meningitis; three cases of bacterial meningitis (Streptococcus mitis, Borrelia burgdorferi (Lyme disease), Staphylococcus epidermidis); a case of cryptococcal meningitis; one case of anti-acetylcholine ganglionic neuronal receptor autoimmune encephalopathy; and a WHO grade III malignant astrocytic neoplasm.

This initial assessment of the entire data set with the heat map and dendrogram supported further investigation of the hypothesis that CNS diseases can be partitioned based on a composite cytokine innate immune profile in a reproducible manner. For example, all six CNS lymphoma cases were present in the same class as 11 of the 12 autoimmune/DM cases, along with all of the severely immunosuppressed patients with CNS infections. The grouping of a disease type within a class confirmed that the innate immune response, as reflected by cytokine levels, is relatively similar within a disease class. Additionally, the grouping of more than one disease type within a given class suggested that certain disease states may share some characteristics of innate immune response (i.e. CNS lymphomas and autoimmune/DM disorders).

### Example 2

### Determination of a Panel of Informative Cytokines that Reveal a Pronounced Separation of CNS Disease Class

In order to achieve the most efficient differentiation of CNS diseases based on CSF cytokine levels, a combination of statistical methods were used to identify a panel of informative cytokines which allow for a more precise separation of the different disease states. Using all 41 cytokines, discriminant analysis was applied. Discriminant analysis is a method used to statistically verify whether groups (here, CNS disease states) can be classified based on measured characteristics (here, cytokines). It can also be used to isolate the variables which have the greatest impact on the separation of the groups. Based on the application of a chi square test, the p-value generated for each cytokine (variable) signified its contribution to the separation of the cases and the groups of diseases.

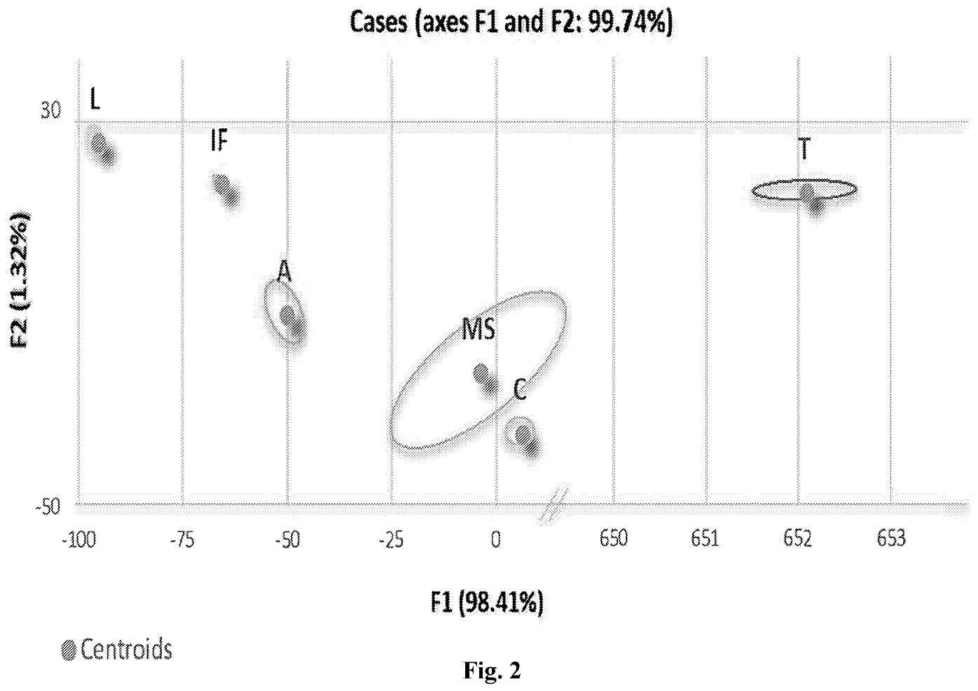

According to discriminant analysis, 100% of the 43 cases were assigned appropriately to their respective disease groups (infection, autoimmune, demyelination (DM), tumor, lymphoma, and control). FIG. 2 shows the observations plotted on the factor axes. The plot shows the groups' centroids with a surrounding ring demonstrating the distribution of the observations within each disease group. This plot demonstrates that CNS diseases separate well based on the cytokine expression.

Utilizing a combination of the aforementioned methods, a panel of cytokines was selected from the initial 41 cytokines: EGF, MDC/CCL22, PDGF-AA, Fractalkine/CX3CL1, IFN-γ, GRO/CXCL1, IL-1β, IL-2, IL-7, IL-8, IL-9, IP-10/CXCL10, TGF-α, IL12-p40, IL12-p70, IL13, IL-15, and TNF-β.

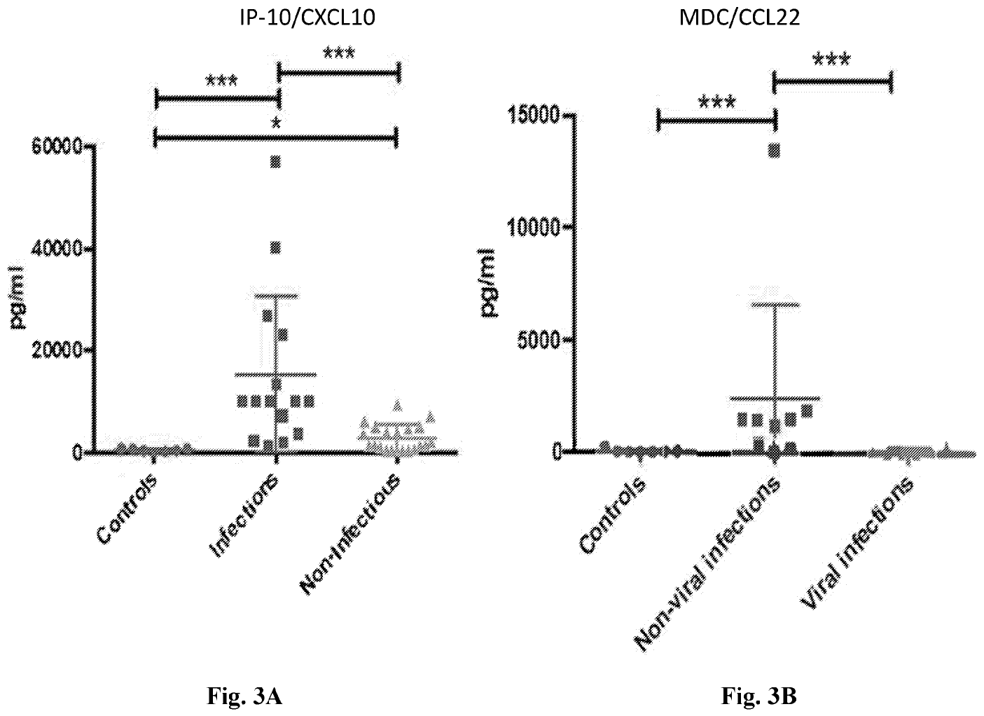

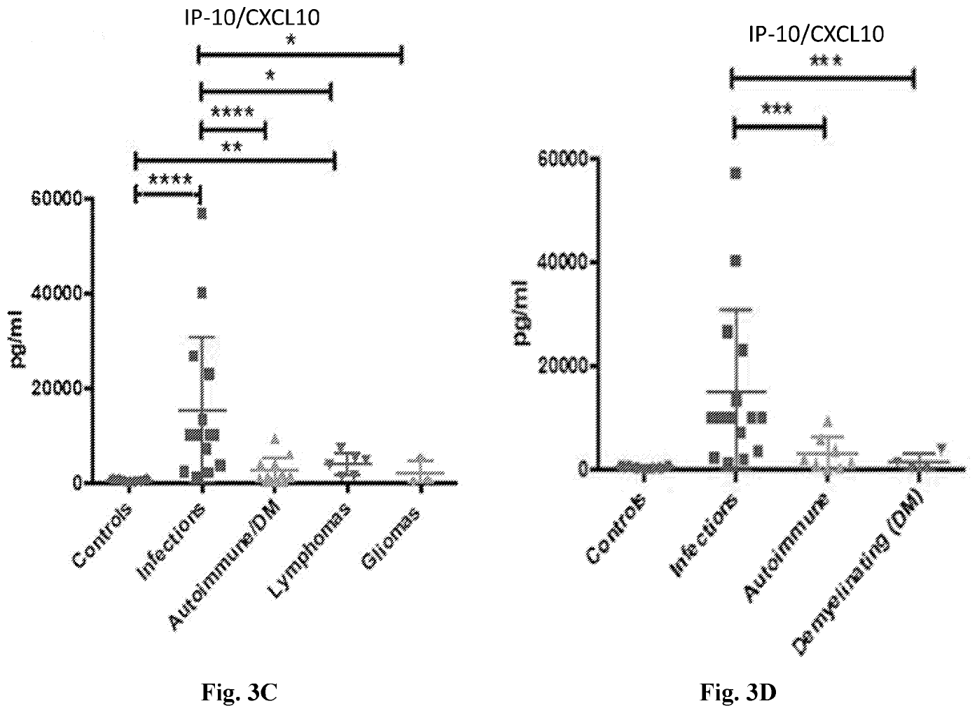

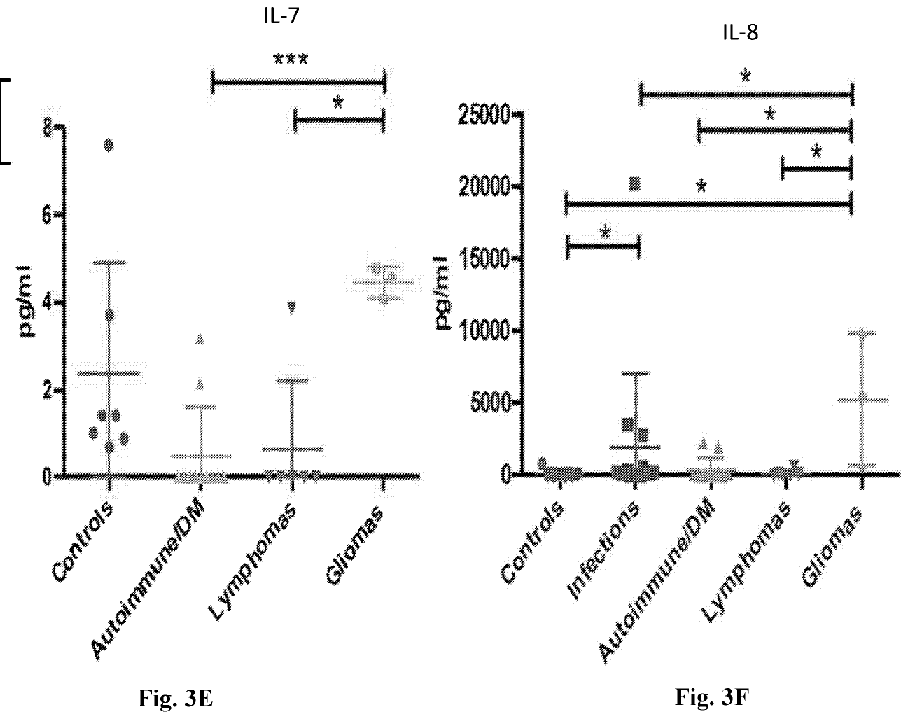

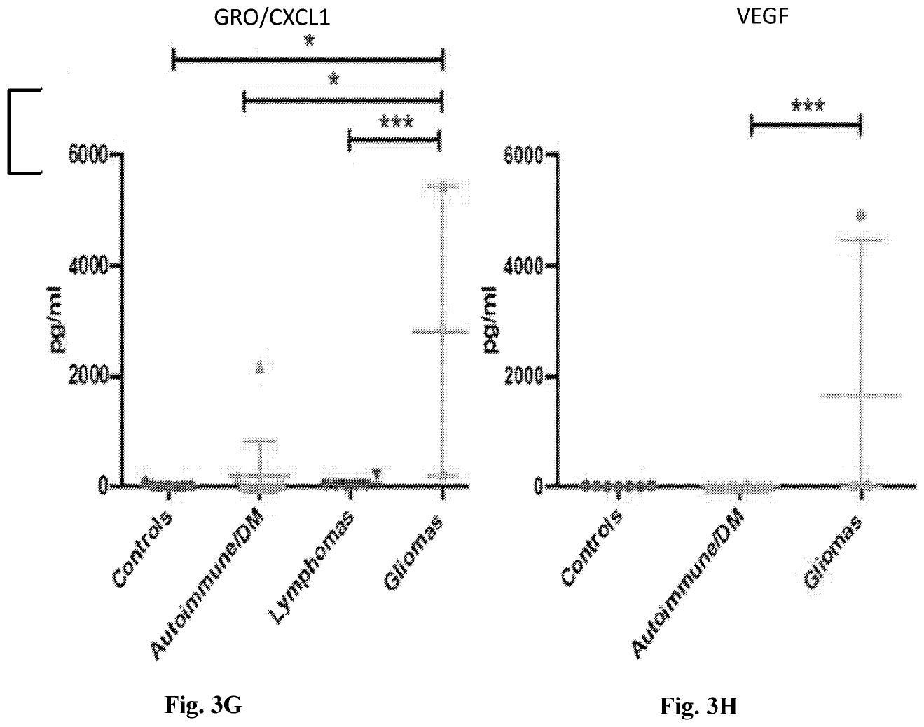

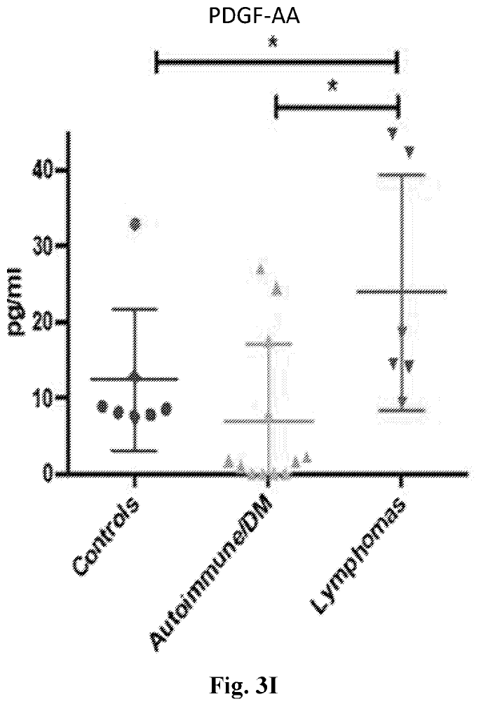

To assess the potential utility of quantifying levels of individual CSF cytokines in distinguishing between distinct CNS diseases, the Mann-Whitney test of significance was used to test for statistical significance in cytokine levels between various CNS disease groups (FIG. 3). IP-10/CXCL10 levels were significantly higher in the pooled infectious cases compared to the pooled non-infectious cases (p<0.0001) and controls (p<0.0001) (FIG. 3A). Infections of various types are pooled in the analysis, since a variety of CNS infections can present to clinical attention with indistinguishable symptoms and similar results in initial testing.

Two critical questions to be answered in the clinical setting that represented major branch points in the clinical decision making process are as follows: “Is this disease an infection?” and “If the disease is an infection, is the pathogen a virus or a non-viral pathogen?” Analysis of IP-10 levels (FIG. 3A) provided information relative to the likelihood of whether the process is an infection. Within the infectious group, MDC/CCL22 levels were significantly higher in non-viral infections compared to viral infections (p=0.0048) and controls (p=0.0012) (FIG. 3B). Thus, CSF measurement of IP-10/CXCL10 levels may be useful in identifying a CNS disease state as suspicious for infection with further stratification of the disease using MDC/CCL22 levels into viral versus non-viral infection subtypes. Levels of IP-10/CXCL10 were also significantly higher in the infection group when compared to the specific autoimmune/DM disease cases (p=0.0005), lymphoma (p=0.0487), and glioma (p=0.0294) groups, and IP-10/CXCL10 levels were significantly higher in infectious and lymphomas compared to controls (p<0.0001 and p=0.0012, respectively) (FIG. 3C). Elevated levels of IP-10/CXCL10 were also significantly higher in infectious cases than both autoimmune cases and demyelinating cases when these two groups were analyzed separately (FIG. 3D).

Interrogation of other cytokines seen in FIG. 3 demonstrated significant differences between non-infectious disease states to potentially contribute to the characterization of the cases not suspected of being infectious. IL-7, IL-8, GRO/CXCL1 and VEGF were informative in distinguishing WHO grade III and IV gliomas from the other disease states studied (FIG. 3E-H). IL-7 levels were significantly higher in gliomas compared to autoimmune/DM cases (p=0.0035) and lymphomas (p=0.0119). Gliomas also displayed higher IL-8 levels when compared to infections (p=0.0392), autoimmune/DM cases (p=0.0176), lymphomas (p=0.0460), and controls (p=0.0333). GRO/CXCL1 levels were higher in gliomas compared to autoimmune/DM (p=0.0044), lymphomas (p=0.0476) and controls (p=0.0167). Higher levels of VEGF are seen in gliomas compared to autoimmune/DM cases (p=0.0286). While lymphomas and autoimmune/DM cases appear as a single class on AHC, PDGF-AA levels prove helpful in separating these two disease groups with CSF from patient with lymphomas having significantly higher levels than autoimmune/DM cases (p=0.0130) and controls (p=0.0221) (FIG. 31). For the analytes presented in FIG. 3 (IP-10/CXCL10, MDC/CCL2, IL-7, IL-8, GRO/CXCL1, VEGF and PDGF-AA), a level of significance of p<0.05 or smaller signifies an observed power of greater than 50%.

### Example 3

### ROC and PCA Analysis Demonstrates Strong Potential of Cytokine Levels for Distinguishing CNS Disorders

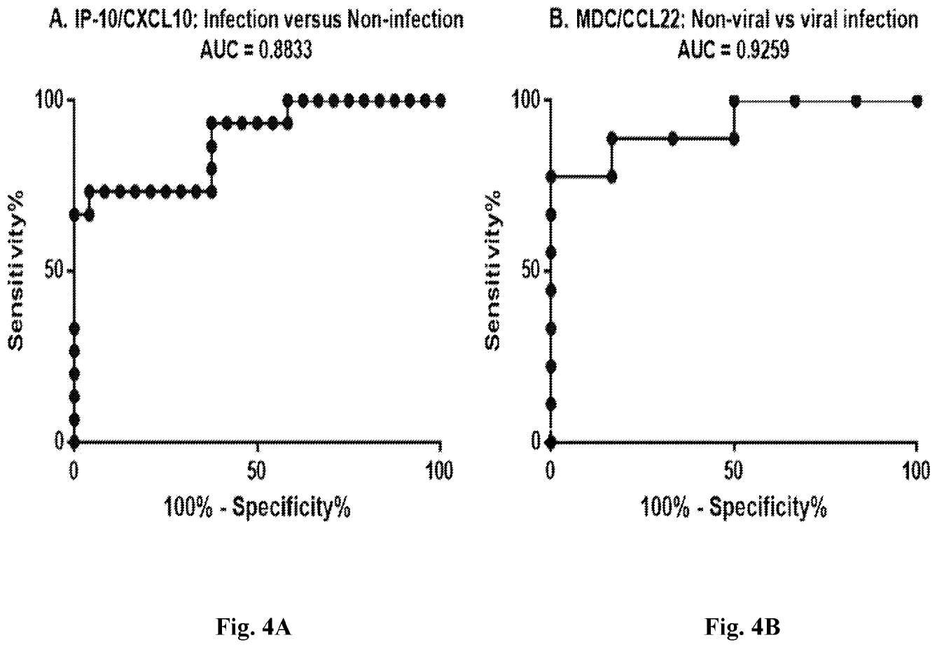

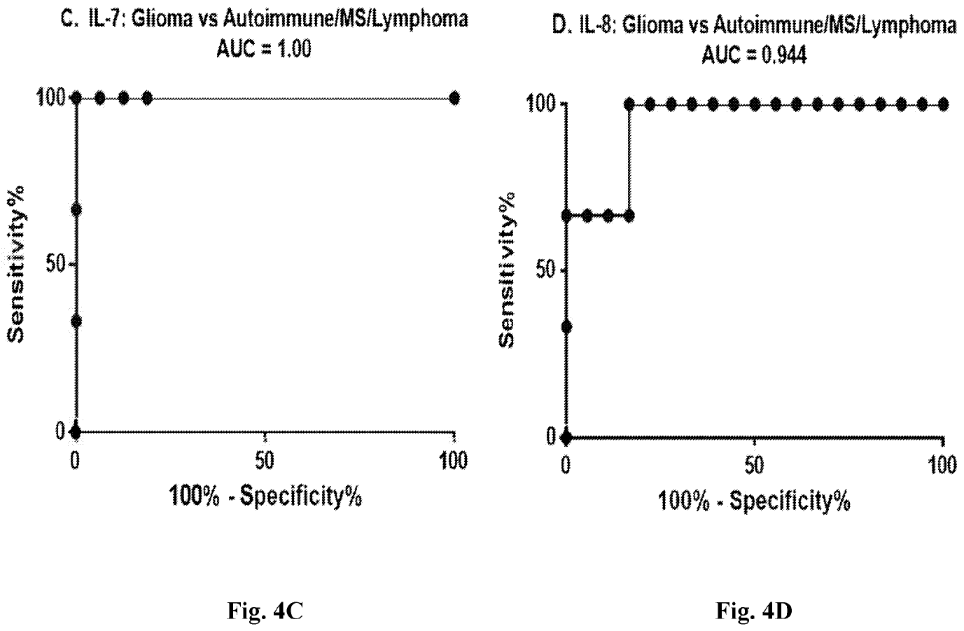

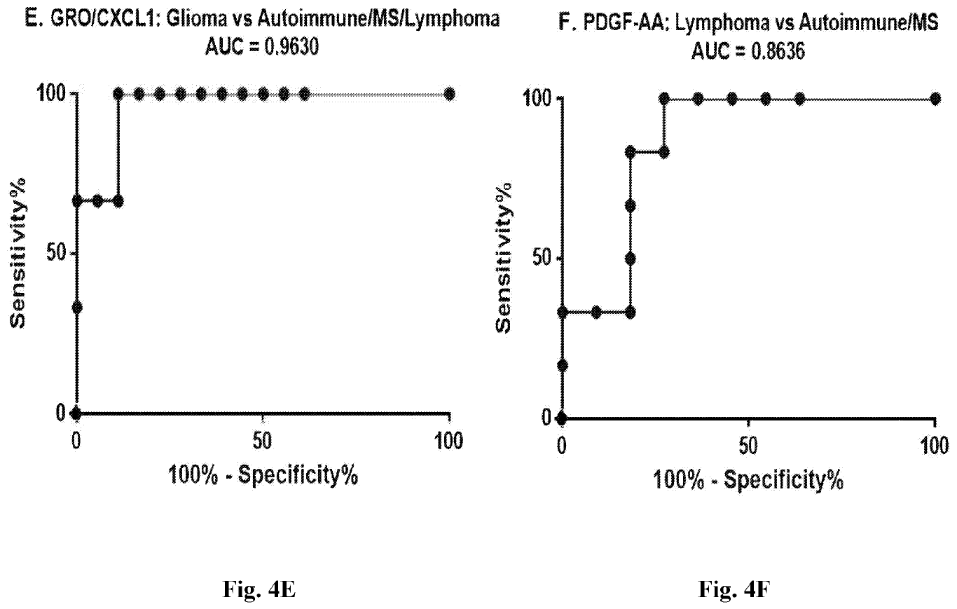

Receiver operator characteristic (ROC) curve analysis is a tool to explore the inherent utility of a method or assay as a diagnostic test. Here, ROC was used to interrogate the potential utility of the above cytokines as individual tests. ROC curves with the corresponding AUC for IP-10/CXCL10, PDGF-AB/BB, IL-7, IL-8, GRO/CXCL1, and PDGF-AA are shown in FIG. 4 (A-F). All of the AUC values ranged between 0.8000 and 1. AUC values in this range are considered to be in either the good (0.8-0.9) or excellent (0.9-1.0) range when grading test adequacy. The results of this analysis support the potential of using levels of these cytokines in CSF to distinguishing different CNS disorders. ROC analysis also suggests analyte cut-off values along with corresponding sensitivities and specificities.

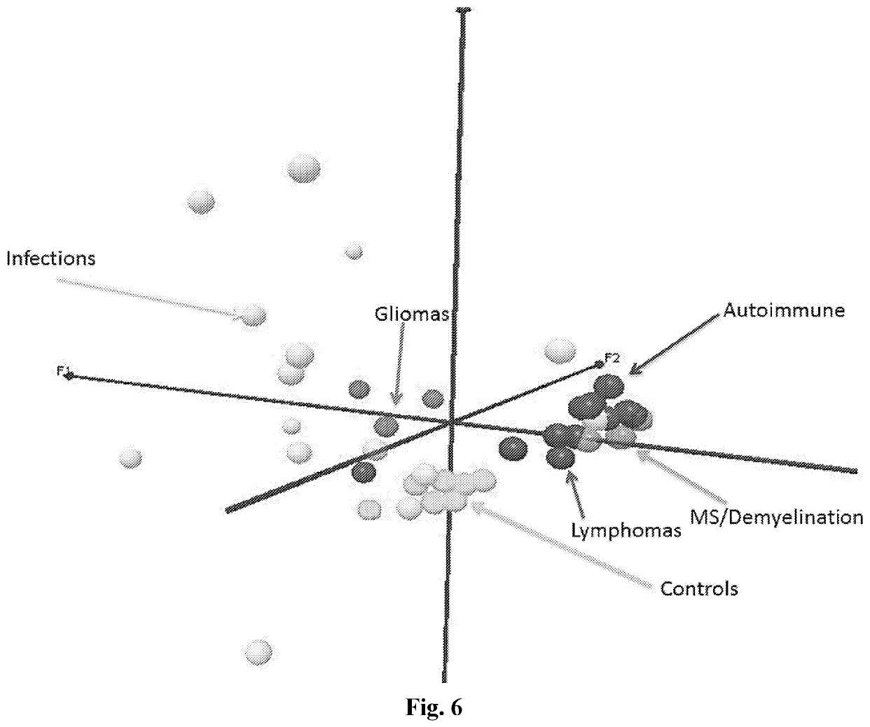

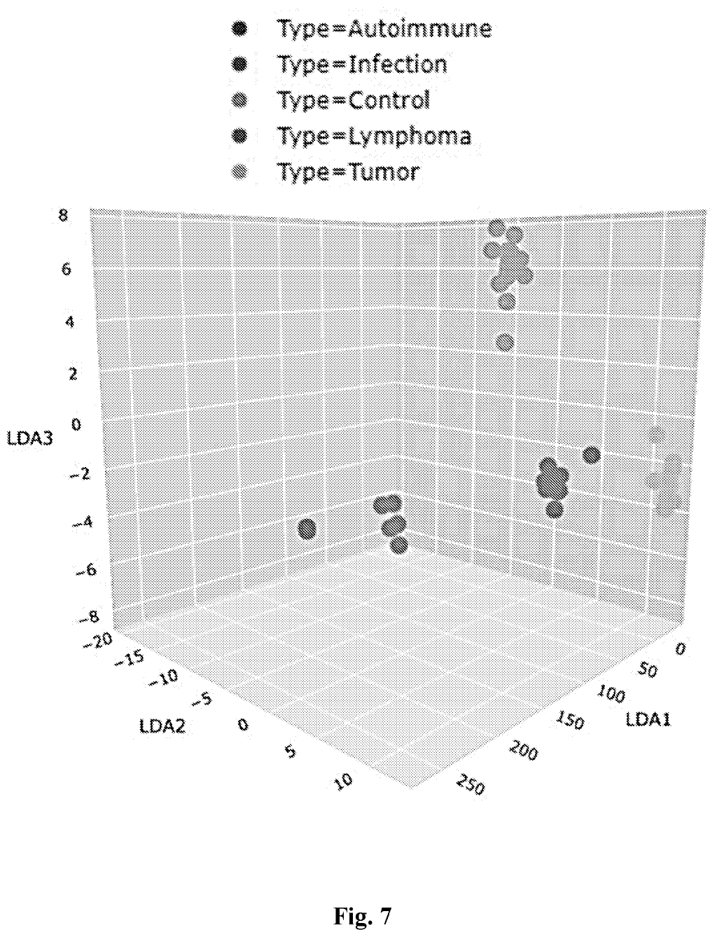

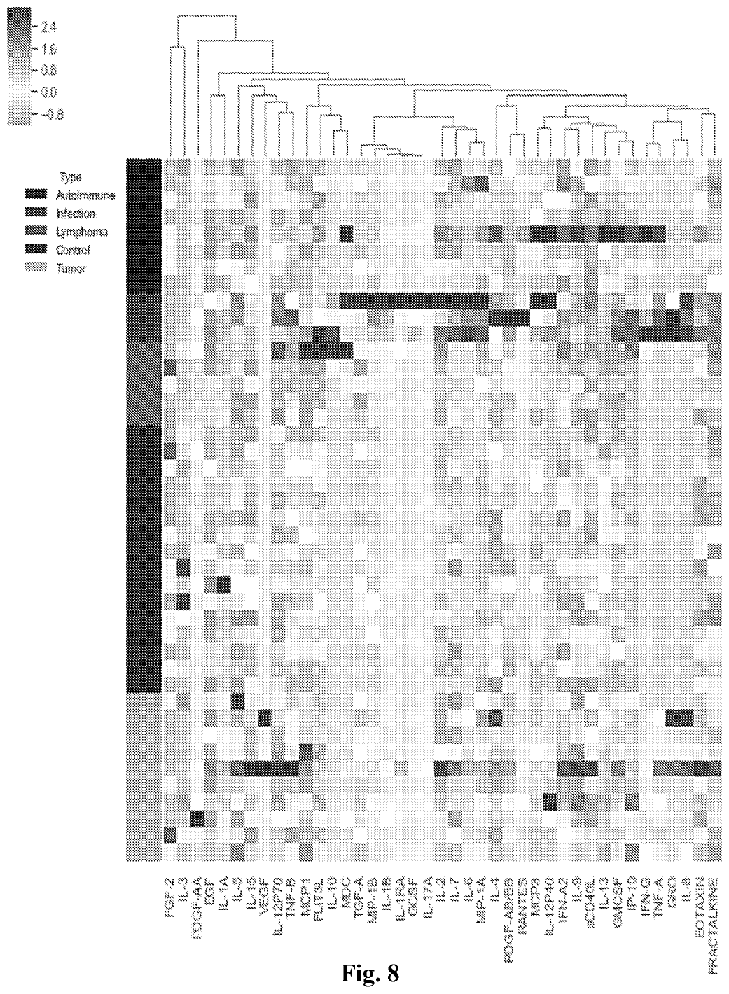

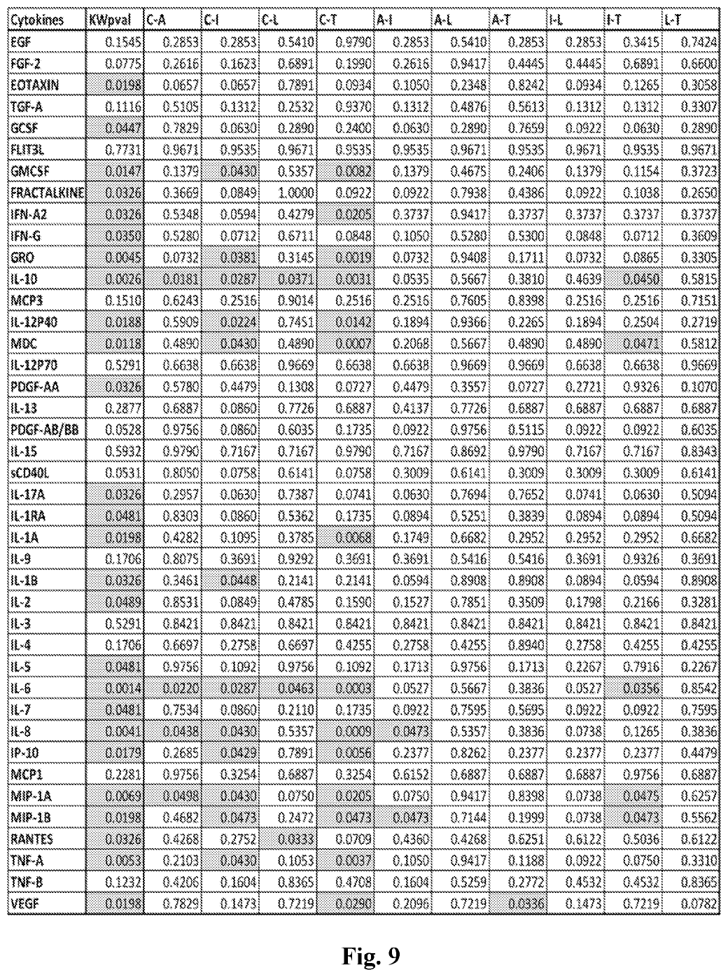

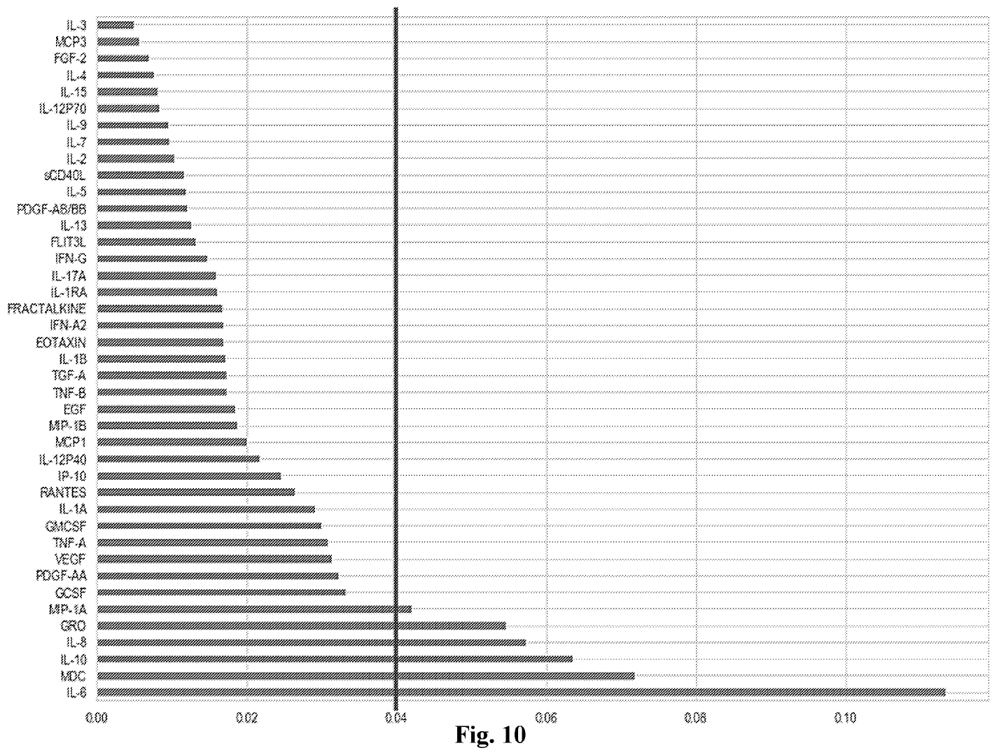

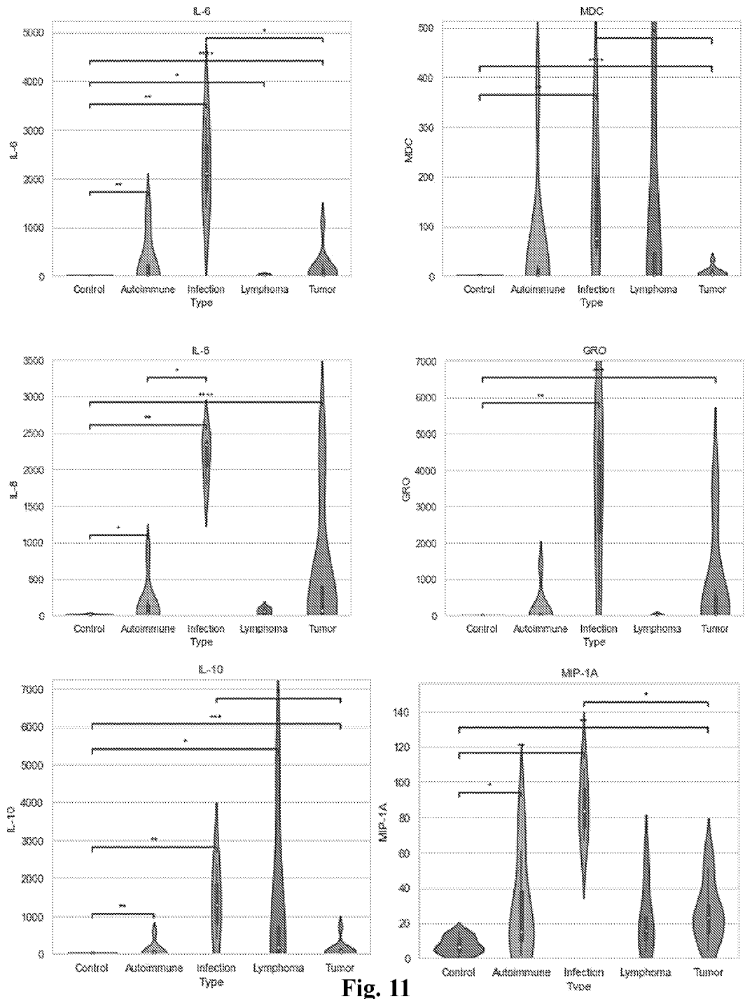

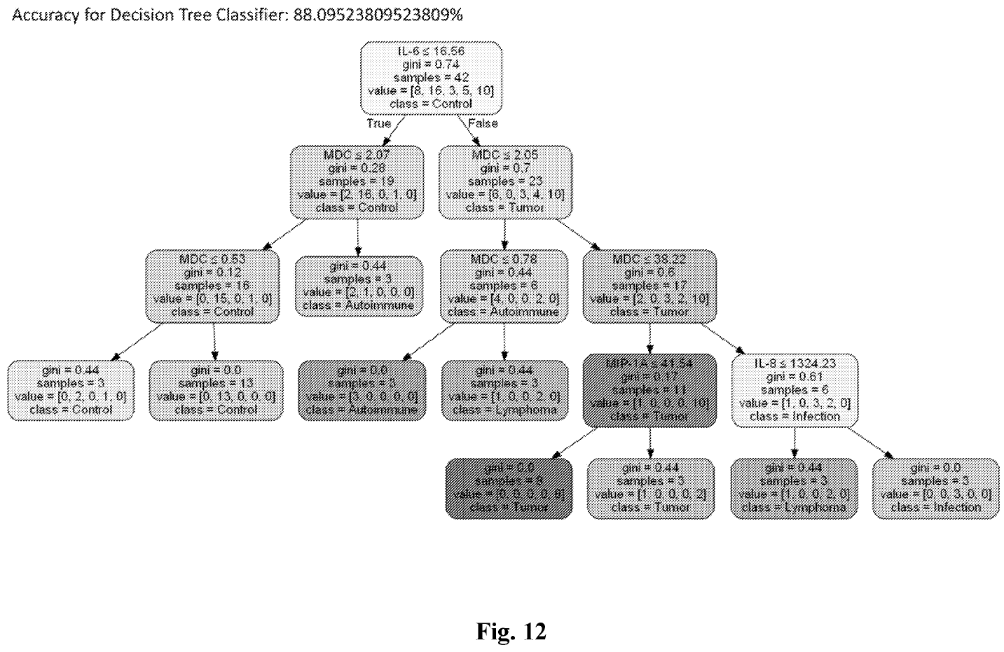

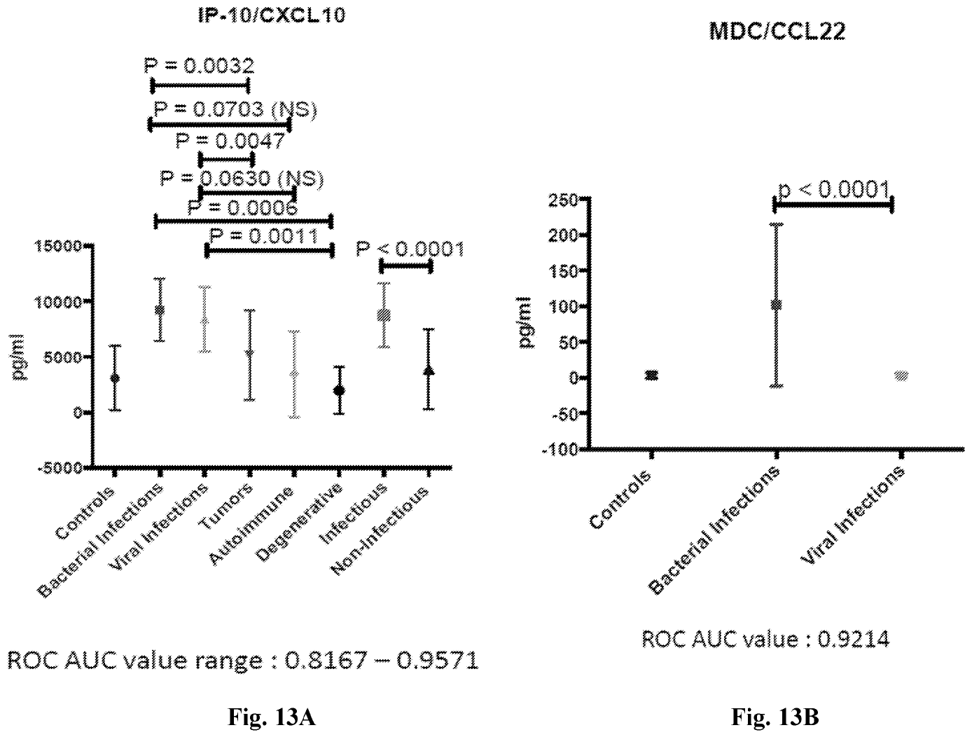

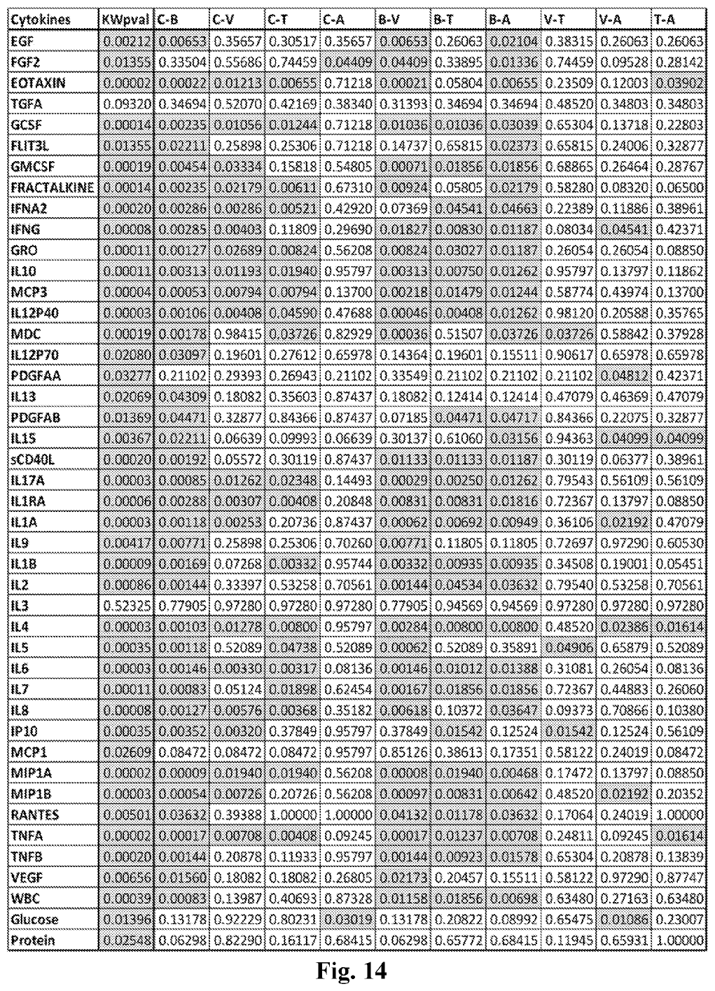

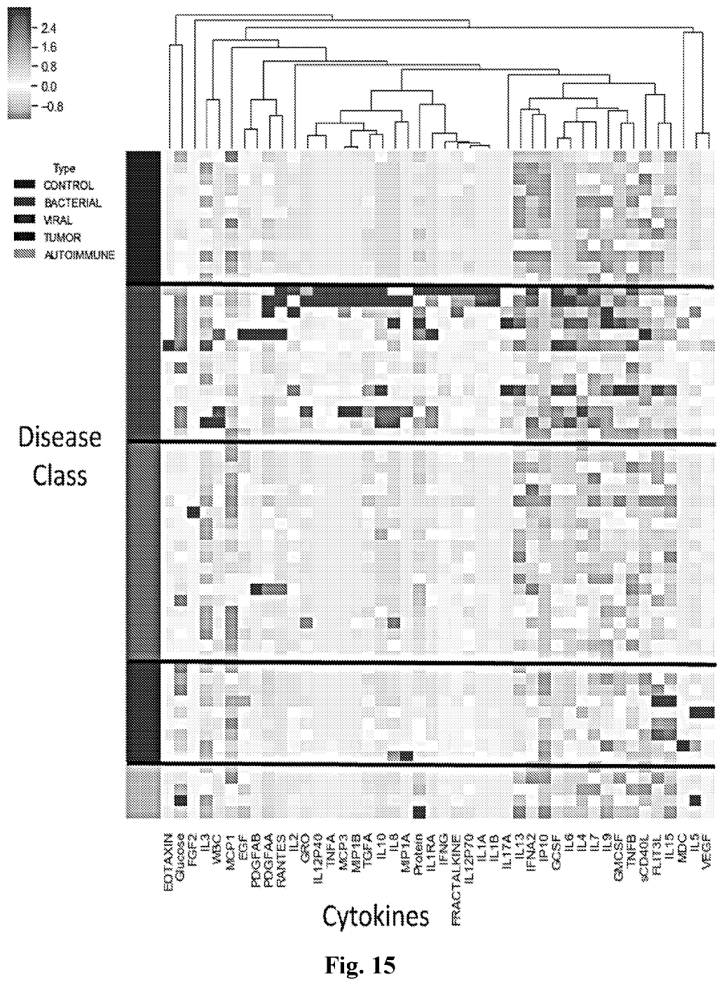

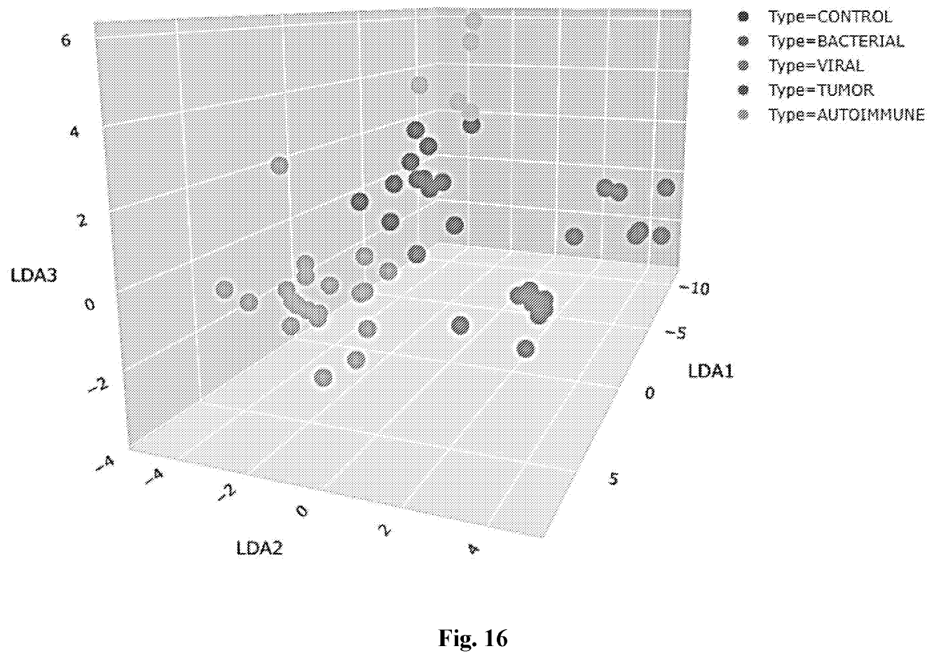

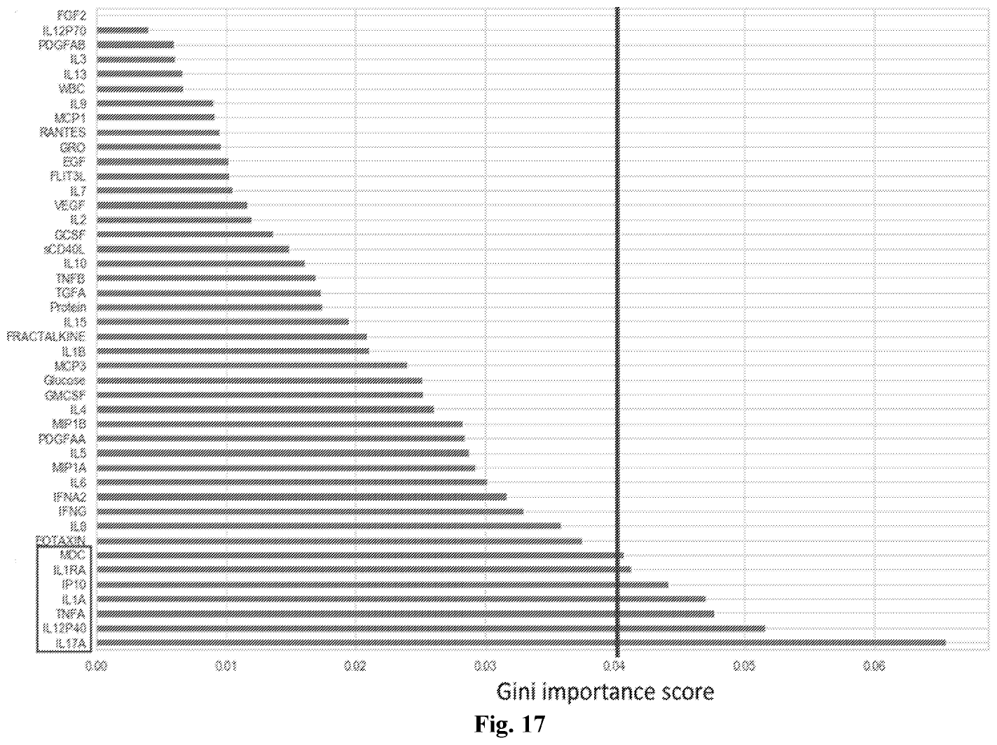

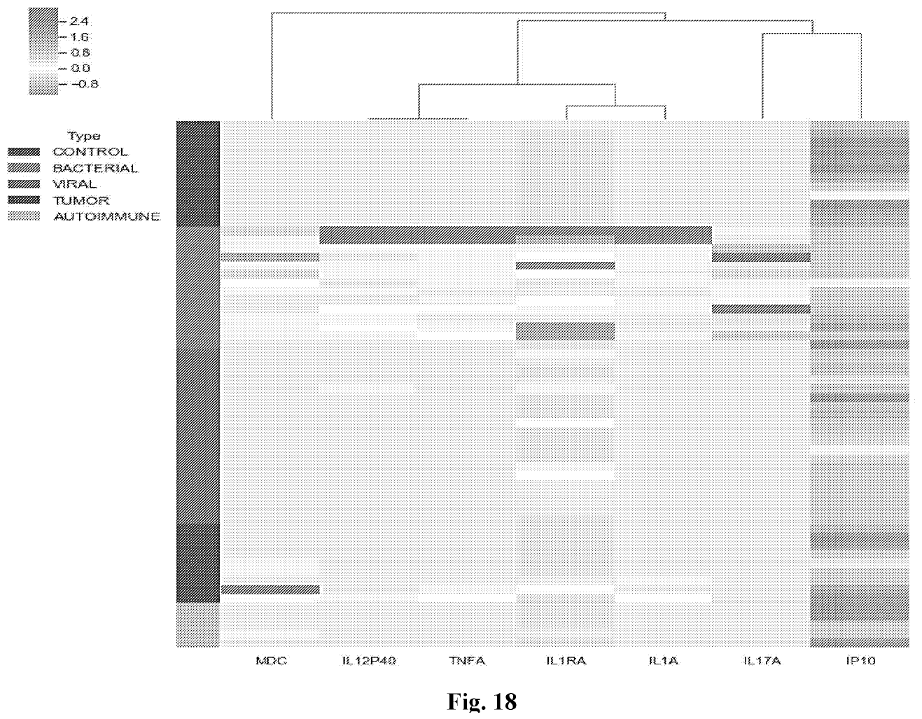

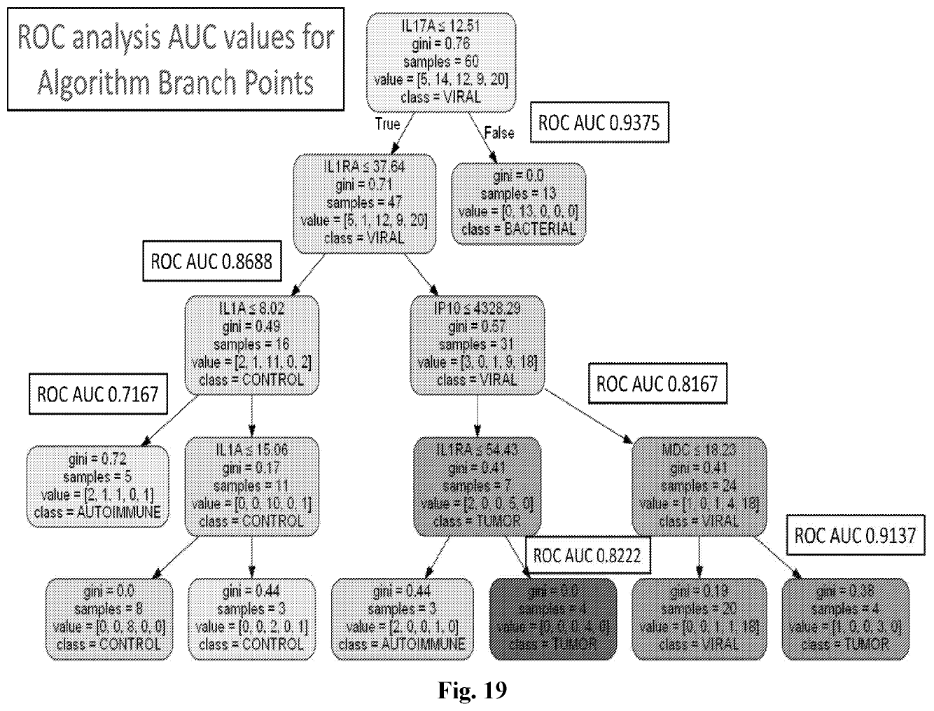

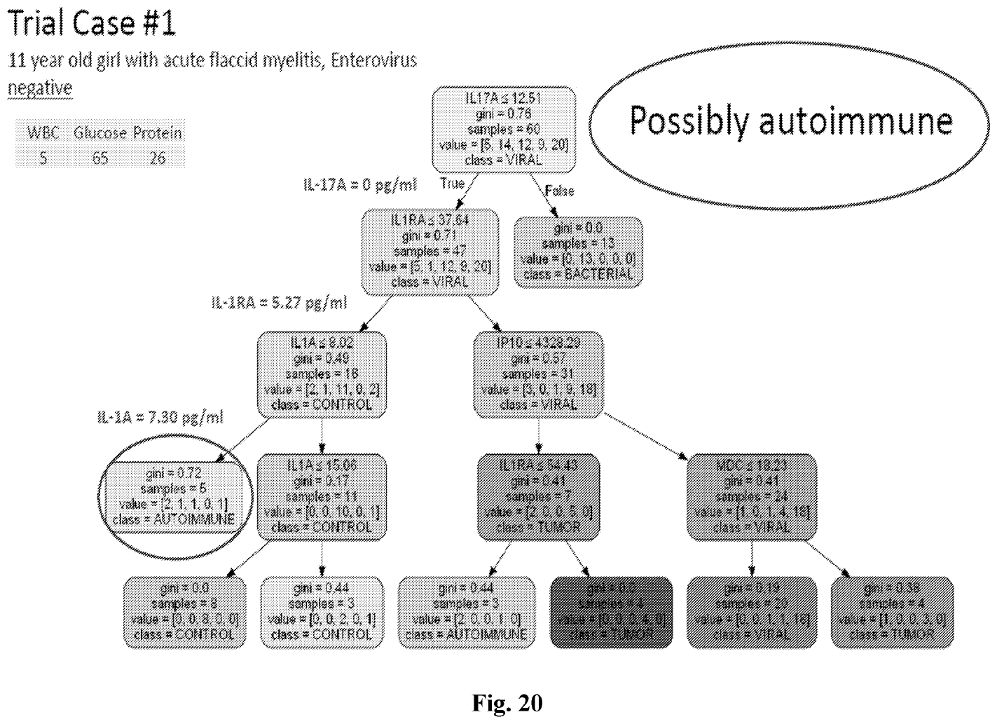

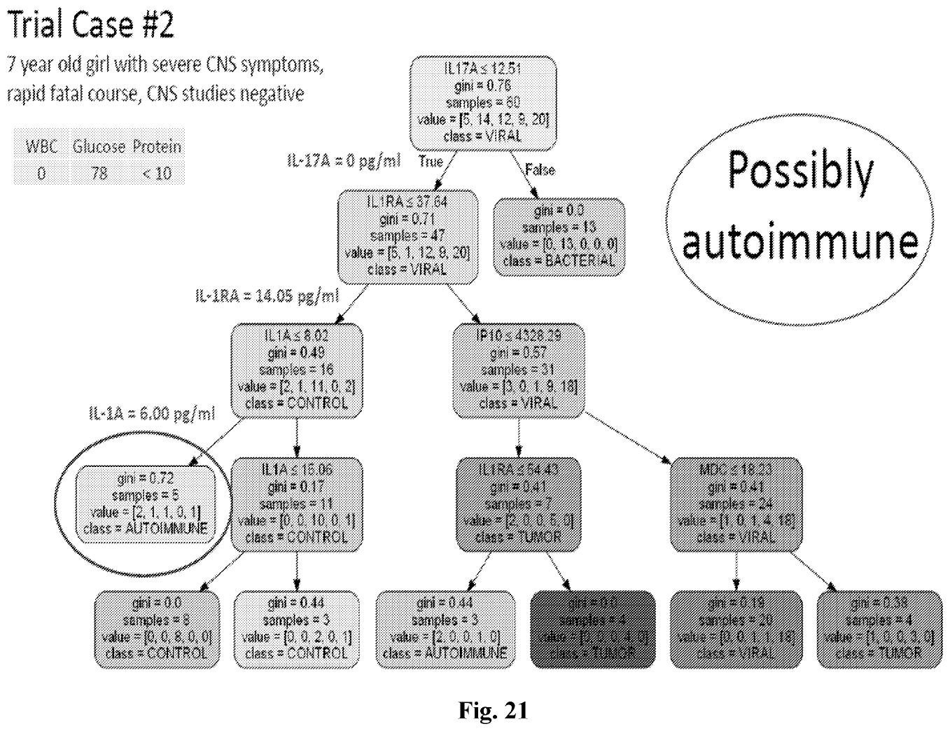

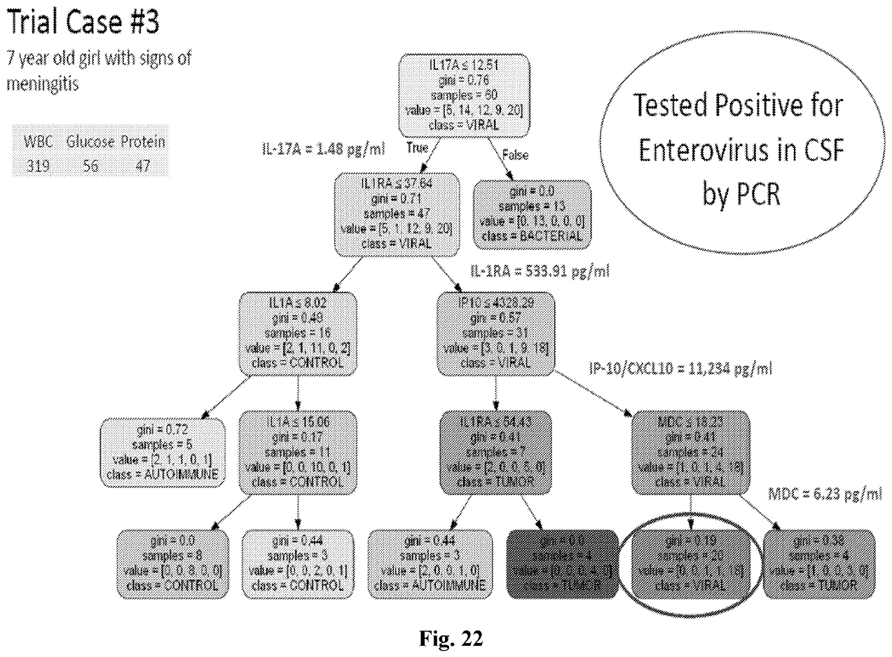

Additionally, principal component analysis (PCA) is another statistical technique used to assess patterns and correlations in a data set. This method transforms a multi-dimensional set of data to a practical dimension for viewing data trends on a plot. PCA differs from the discriminant analysis in that PCA constructs the best clustering and discrimination of the observations without any previous knowledge of any predetermined group allocations. The generated principal components (P1, P2, P3, etc.) are linear representations of the variables (cytokines) which describe the maximum variation in the data set (FIG. 6).

By plotting all cases using coordinates generated by the principal components and the observed values, the multi-dimensional (in the present case 18-dimensional) data set can be visualized on a three-dimensional plot. Here, approximately 70% of the variance in the data can be explained by the first three principal components. While a common use of PCA is to demonstrate similarity of observations and of variables as points on maps as a step in the validation of methods, a PCA plot was generated using the original data set to demonstrate what analysis of a larger data set yields during validation of the present approach (FIG. 6).

### Example 4

### Establishing a Diagnostic Algorithm for CNS Disease

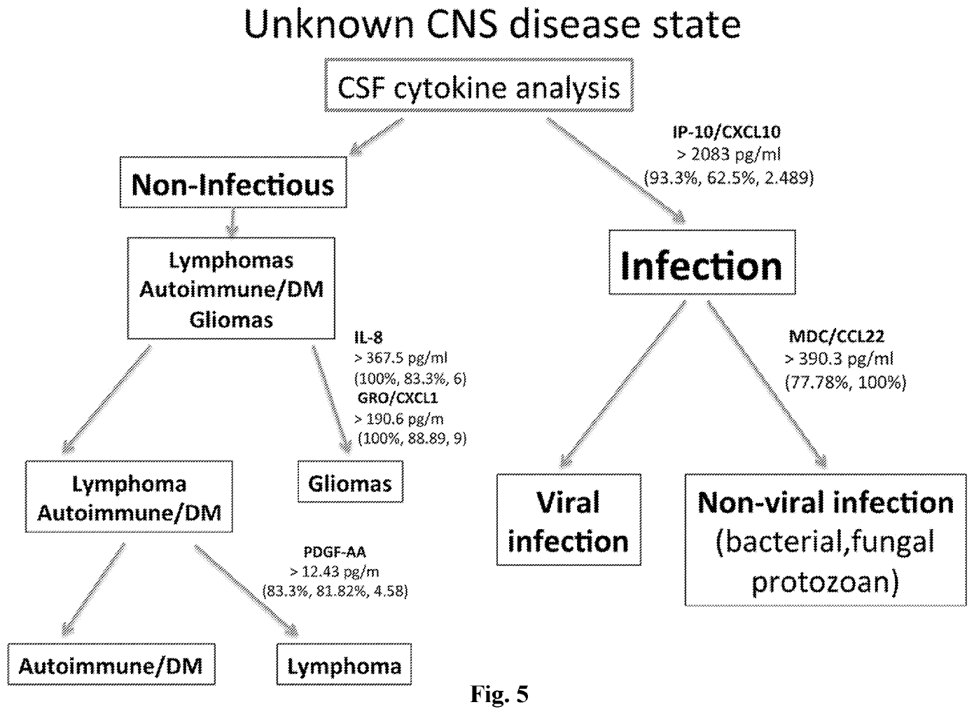

Based on the cut-off values suggested by ROC, a prototype diagnostic algorithm flowchart was constructed (FIG. 5) using the different CSF cytokine levels to identify probable infectious cases, sub-classify them as viral or non-viral, and suggest the nature of the non-infectious cases. The initial data set revealed the importance of IP-10/CXCL10 and MDC in identifying infectious from non-infectious CNS disorders and in distinguishing viral CNS infections from CNS infections caused by non-viral pathogens. Application of other statistical analyses on additional data sets, further validated the role CSF cytokine profiles play in identifying CNS disease type (See Example 5 and Example 6).

### Example 5

### Machine Learning Generated Informative Cytokines Useful in Identifying CNS Disease Type in Adults

The clinical presentation and imaging features of certain types of central nervous system (CNS) disorders often overlap, significantly delaying appropriate therapy. Although they are useful in identifying CNS bacterial infections, routine cerebral spinal fluid (CSF) white blood cell (WBC) count, protein, and glucose levels are often inadequate to discriminate CNS viral infections, autoimmune diseases, lymphomas, and tumors. This inability to rapidly discriminate among CNS disease types not only leads to excessive laboratory testing but may also result in the initiation of inappropriate therapy. Here the potential of using CSF cytokines to discriminate patients with tumors, lymphomas, autoimmune diseases, and infections in the CNS was examined. Levels of 41 CSF cytokines were quantified in adult patients with CNS tumors (n=10), autoimmune diseases (n=8), lymphomas (n=5), infections (n=3), and systemic disease without CNS involvement (n=15). Agglomerative hierarchical clustering (AHC) and linear discriminant analysis (LDA) were used to demonstrate whether CSF cytokine levels could distinguish samples by CNS disease type. The Kruskal-Wallis and post-hoc Mann-Whitney tests were used to determine significant differences. Unbiased random forest machine learning then selected cytokines with the highest ability to discriminate the CNS disease classes. All statistical calculations in this analysis are performed using Python packages.

The accuracy of a decision tree built with the selected cytokines was compared to a tree constructed using routine CSF values. An AHC generated heat map demonstrated distinct cytokine profiles for the different CNS disease types. LDA revealed robust cytokine-dependent patient sample clustering by CNS disease type. Of the 41 cytokines analyzed, 24 showed significant differences among the CNS disease classes. An unbiased algorithm then identified five cytokines (IL-6, IL-10, IL-8, MDC, and GRO) as having the highest discriminatory power among the different CNS disease types. A decision tree with these cytokines significantly outperformed a tree using routine CSF values (accuracy 82.9% vs 65.8%). These results demonstrated that CSF cytokine profiles of various CNS disease are distinct and that CSF cytokine-based algorithms can rapidly identify the class of CNS disease afflicting patients.

### Example 6

### Pediatric CSF Cytokine Profile Utility in Distinguishing CNS Disorders in Infants and Children

Rapid characterization of CNS diseases in pediatric patients is a necessary step for initiating timely and proper care. Even using current technologies, determining if the CNS is involved, and if so, characterizing the type of CNS disease is often difficult in the acute setting. Novel diagnostic approaches to quantify the immune response elicited by different CNS disorders may contribute to the rapid determination of CNS disease class. Examination of CSF cytokine levels in pediatric patients to identify cytokine profiles useful in distinguishing CNS bacterial infections, viral infections, primary tumors, autoimmune diseases, and systemic disease without CNS involvement was performed. 41 CSF cytokine levels were quantified in pediatric patients (ages 11 days-14 years) with CNS bacterial infections (n=14), viral infections (n=20), autoimmune diseases (n=5), primary tumors (n=9), and controls without CNS involvement (n=12). Routine CSF values (protein, glucose, and white blood cell counts) were also measured. Linear discriminant analysis (LDA) was used to demonstrate if CSF cytokines can be used to cluster samples by CNS disease type. The Kruskal-Wallis and post-hoc Mann-Whitney tests were used to determine significant differences. Unbiased random forest machine learning then selected cytokines with the highest ability to discriminate the CNS disease classes. All statistical calculations in this analysis are performed using Python packages. Finally, the accuracy of a decision tree built with the selected cytokines was compared to a tree constructed using routine CSF values. LDA showed that CSF cytokines can distinctly cluster patient samples by CNS disease type. Of the 41 cytokines analyzed, 39 showed significant differences among the CNS disease classes. An unbiased algorithm then identified 7 cytokines (IL17A, IL12p40, TNFA, IL1A, IP10, IL1RA, and MDC) as having the highest discriminatory power among the different CNS disease types. A decision tree utilizing these informative cytokines significantly outperformed a tree based on routine CSF values (accuracy 86.7% vs. 76.7%). Results showed that CSF cytokine profiles in pediatric patients afflicted with different types of CNS disease are distinct. Cytokine profile based characterization of CNS disease type was robust. In this example, it was demonstrated that CSF cytokine profiles of various CNS diseases are distinct and that CSF cytokine-based algorithms can rapidly identify the class of CNS disease afflicting pediatric patients.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating a Central Nervous System (CNS) disorder in a pediatric patient in need thereof, the method comprising:
measuring cytokine levels in a cerebrospinal fluid (CSF) sample from the pediatric patient, and comparing the patient sample levels to a first reference sample,
wherein when the level of IL17A is lower in the patient sample compared to the reference sample, the CNS disorder is not a bacterial infection, and wherein when the level of IL17A is higher in the patient sample compared to the reference sample, the CNS disorder is a bacterial infection and an anti-bacterial treatment is administered to the patient, and
wherein the cytokine levels are measured using a technology selected from the group consisting of the Luminex FlexMPA 3D technology, microarray, sequencing, ELISA, and qPCR.

2. The method of claim 1, wherein when the CNS disorder is not a bacterial infection, and the level of IL1RA is lower in the patient sample compared to the reference sample, and the level of IL1A is lower in the patient sample compared to the reference sample, the CNS disorder is an autoimmune disorder and a treatment for an autoimmune disorder is administered to the patient.

3. The method of claim 1, wherein when the CNS disorder is not a bacterial infection, and the level of IL1RA is higher in the patient sample compared to the reference sample, and the level of IP10 is lower in the patient sample compared to the reference sample, and the level of IL1RA is lower in the patient sample compared to a second reference sample, the CNS disorder is an autoimmune disorder and a treatment for an autoimmune disorder is administered to the patient.

4. The method of claim 1, wherein when the CNS disorder is not a bacterial infection, and the level of IL1RA is higher in the patient sample compared to the reference sample, and the level of IP10 is lower in the patient sample compared to the reference sample, and the level of IL1RA is higher in the patient sample compared to a second reference sample, the CNS disorder is a cancer and a treatment for cancer is administered to the patient.

5. The method of claim 1, wherein when the CNS disorder is not a bacterial infection, and the level of IL1RA is higher in the patient sample compared to the reference sample, and the level of IP10 is higher in the patient sample compared to the reference sample, and the level of MDC is lower in the patient sample compared to the reference sample, the CNS disorder is viral and an anti-viral treatment is administered to the patient.

6. The method of claim 1, wherein when the CNS disorder is not a bacterial infection, and the level of IL1RA is higher in the patient sample compared to the reference sample, and the level of IP10 is higher in the patient sample compared to the reference sample, and the level of MDC is higher in the patient sample compared to the reference sample, the CNS disorder is a cancer and a treatment for cancer is administered to the patient.

7. A method of treating a Central Nervous System (CNS) disorder, the method comprising:
measuring cytokine levels in a cerebrospinal fluid (CSF) sample from a patient and comparing the patient sample levels to a reference sample,
wherein when the level of IP-10/CXCL10 is lower in the patient sample compared to the reference sample, the CNS disorder is non-infectious, and
wherein when the level of IP-10/CXCL10 is higher in the patient sample compared to the reference sample, the CNS disorder is infectious, and a treatment is administered to the patient that treats the infection, and
wherein the cytokine levels are measured using a technology selected from the group consisting of the Luminex FlexMPA 3D technology, microarray, sequencing, ELISA, and qPCR.

8. The method of claim 7, further comprising wherein when the CNS disorder is infectious and the level of MDC/CCL22 is higher in the patient sample compared to the reference sample, the CNS disorder is a non-viral disorder, and an anti-bacterial and/or anti-fungal and/or anti-parasitic treatment is administered to the patient, and
wherein when the level of MDC/CCL22 is lower in in the patient sample compared to the reference sample, the CNS disorder is a viral disorder, and an anti-viral treatment is administered to the patient.

9. The method of claim 7, further comprising wherein when the CNS disorder is non-infectious, and the levels of IL-8 and GRO/CXCL1 are higher in the patient sample compared to the reference sample, the CNS disorder is a glioma, and a treatment for gliomas is administered to the patient, and
wherein when the level of IL-8 and GRO/CXCL1 are lower in the patient sample compared to the reference sample, the CNS disorder is an autoimmune disorder or a lymphoma, and a treatment for an autoimmune disorder or a lymphoma is administered to the patient.

10. The method of claim 7, further comprising wherein when the CNS disorder is an autoimmune disorder or a lymphoma, and wherein when the level of PDGF-AA is higher in the patient sample compared to the reference sample, the CNS disorder is a lymphoma, and an treatment for lymphomas is administered to the patient, and wherein when the level of PDGF-AA is lower in the patient sample compared to the reference sample, the CNS disorder is an autoimmune disorder and a treatment for an autoimmune disorder is administered to the patient .

11. A method of treating a Central Nervous System (CNS) disorder, the method comprising:
measuring cytokine levels in a cerebrospinal fluid (CSF) sample from a patient and comparing the patient sample levels to a reference sample,
wherein when the level of IL-6 is lower in the patient sample compared to the reference sample, and the level of MDC is higher in the patient sample compared to the reference sample, the CNS disorder is an autoimmune disorder and a treatment for an autoimmune disorder is administered to the patient, and
wherein the cytokine levels are measured using a technology selected from the group consisting of the Luminex FlexMPA 3D technology, microarray, sequencing, ELISA, and qPCR.

12. A method of treating a Central Nervous System (CNS) disorder, the method comprising:
measuring cytokine levels in a cerebrospinal fluid (CSF) sample from a patient and comparing the patient sample levels to a first, second, and third reference sample,
wherein when the level of IL-6 is higher in the patient sample compared to the first reference sample, and the level of MDC is lower in the patient sample compared to the second reference sample and compared to the third reference sample, the CNS disorder is an autoimmune disorder and a treatment for an autoimmune disorder is administered to the patient, and
wherein the cytokine levels are measured using a technology selected from the group consisting of the Luminex FlexMPA 3D technology, microarray, sequencing, ELISA, and qPCR.

13. A method of treating a Central Nervous System (CNS) disorder, the method comprising:
measuring cytokine levels in a cerebrospinal fluid (CSF) sample from a patient and comparing the patient sample levels to a first, second, and third reference sample,
wherein when the level of IL-6 is higher in the patient sample compared to the first reference sample, and the level of MDC is lower in the patient sample compared to the second reference sample but higher compared to the third reference sample, the CNS disorder is a lymphoma and a treatment for lymphoma is administered to the patient, and
wherein the cytokine levels are measured using a technology selected from the group consisting of the Luminex FlexMPA 3D technology, microarray, sequencing, ELISA, and qPCR.

14. A method of treating a Central Nervous System (CNS) disorder, the method comprising:
measuring cytokine levels in a cerebrospinal fluid (CSF) sample from a patient and comparing the patient sample levels to a first, second, third, and fourth reference sample,
wherein when the level of IL-6 is higher in the patient sample compared to the first reference sample, and the level of MDC is higher in the patient sample compared to the second reference sample but lower compared to the third reference sample, and the level of MIP-1A is higher in the patient sample compared to the fourth reference sample, the CNS disorder is a cancer and a treatment for cancer is administered to the patient, and
wherein the cytokine levels are measured using a technology selected from the group consisting of the Luminex FlexMPA 3D technology, microarray, sequencing, ELISA, and qPCR.

15. A method of treating a Central Nervous System (CNS) disorder, the method comprising:
measuring cytokine levels in a cerebrospinal fluid (CSF) sample from a patient and comparing the patient sample levels to a first, second, third, and fourth reference sample,
wherein when the level of IL-6 is higher in the patient sample compared to the first reference sample, and the level of MDC is higher in the patient sample compared to the second reference sample but lower compared to the third reference sample, and the level of MIP-1A is lower in the patient sample compared to the fourth reference sample, the CNS disorder is a cancer and a treatment for cancer is administered to the patient, and
wherein the cytokine levels are measured using a technology selected from the group consisting of the Luminex FlexMPA 3D technology, microarray, sequencing, ELISA, and qPCR.

16. A method of treating a Central Nervous System (CNS) disorder, the method comprising:
measuring cytokine levels in a cerebrospinal fluid (CSF) sample from a patient and comparing the patient sample levels to a first, second, third, and fourth reference sample,
wherein when the level of IL-6 is higher in the patient sample compared to the first reference sample, and the level of MDC is higher in the patient sample compared to the second reference sample and the third reference sample, and the level of IL-8 is lower in the patient sample compared to the fourth reference sample the CNS disorder is a lymphoma and a treatment for lymphoma is administered to the patient, and
wherein the cytokine levels are measured using a technology selected from the group consisting of the Luminex FlexMPA 3D technology, microarray, sequencing, ELISA, and qPCR.

17. A method of treating a Central Nervous System (CNS) disorder, the method comprising:
measuring cytokine levels in a cerebrospinal fluid (CSF) sample from a patient and comparing the patient sample levels to a first, second, third, and fourth reference sample,
wherein when the level of IL-6 is higher in the patient sample compared to the first reference sample, and the level of MDC is higher in the patient sample compared to the second reference sample and the third reference sample, and the level of IL-8 is higher in the patient sample compared to the fourth reference sample, the CNS disorder is infectious and an anti-bacterial and/or anti-fungal and/or anti-parasitic treatment and/or anti-viral treatment is administered to the patient, and
wherein the cytokine levels are measured using a technology selected from the group consisting of the Luminex FlexMPA 3D technology, microarray, sequencing, ELISA, and qPCR.

18. (canceled)

19. (canceled)

20. A composition useful for determining whether a CNS disorder in a patient is infectious, wherein the composition comprises an agent capable of binding IP-10/CXCL10, and an agent capable of binding MDC/CCL22 and
wherein the agent is selected from the group consisting of an antibody, a probe, and a nucleotide sequence, and
wherein the agent is bound to a microarray, and
wherein the agent is fluorescently labeled.

21. A composition useful for determining whether a CNS disorder in a patient is a lymphoma, a glioma, or an autoimmune disorder, wherein the composition comprises an agent capable of binding IP-10/CXCL10, an agent capable of binding IL-8, an agent capable of binding GRO/CXCL1, and an agent capable of binding PDGF-AA, and
wherein the agent is selected from the group consisting of an antibody, a probe, and a nucleotide sequence, and
wherein the agent is bound to a microarray, and
wherein the agent is fluorescently labeled.

22. A composition useful for determining whether a CNS disorder in a pediatric patient is an autoimmune disorder, wherein the composition comprises an agent capable of binding IL17A, an agent capable of binding IL1RA, and an agent capable of binding IL1A, and
wherein the agent is selected from the group consisting of an antibody, a probe, and a nucleotide sequence, and
wherein the agent is bound to a microarray, and
wherein the agent is fluorescently labeled.

23. A composition useful for determining whether a CNS disorder in a pediatric patient is an autoimmune disorder, wherein the composition comprises an agent capable of binding IL17A, an agent capable of binding IL1RA, and an agent capable of binding IP10 and
wherein the agent is selected from the group consisting of an antibody, a probe, and a nucleotide sequence, and
wherein the agent is bound to a microarray, and
wherein the agent is fluorescently labeled.

24. A composition useful for determining whether a CNS disorder in a pediatric patient is bacterial, wherein the composition comprises an array comprising an agent capable of binding IL17A and an agent capable of binding IL1RA, and
wherein the agent is selected from the group consisting of an antibody, a probe, and a nucleotide sequence, and
wherein the agent is bound to a microarray, and
wherein the agent is fluorescently labeled.

25. A composition useful for determining whether a CNS disorder in a pediatric patient is viral, wherein the composition comprises an array comprising an agent capable of binding IL17A, an agent capable of binding IL1RA, an agent capable of binding IP 10, and an agent capable of binding MDC, and
wherein the agent is selected from the group consisting of an antibody, a probe, and a nucleotide sequence, and
wherein the agent is bound to a microarray, and
wherein the agent is fluorescently labeled.

26. A composition useful for determining whether a CNS disorder in a pediatric patient is cancerous, wherein the composition comprises an agent capable of binding IL17A, an agent capable of binding IL1RA, and an agent capable of binding IP10, and
wherein the agent is selected from the group consisting of an antibody, a probe, and a nucleotide sequence, and
wherein the agent is bound to a microarray, and
wherein the agent is fluorescently labeled.

27. A composition useful for determining whether a CNS disorder in a pediatric patient is cancerous, wherein the composition comprises an agent capable of binding IL17A, an agent capable of binding IL1RA, an agent capable of binding IP10, and an agent capable of binding MDC, and
wherein the agent is selected from the group consisting of an antibody, a probe, and a nucleotide sequence, and
wherein the agent is bound to a microarray, and
wherein the agent is fluorescently labeled.

28. (canceled)

29. (canceled)

30. (canceled)
